SEQUENCE LISTING (5 sequences)

SEQ ID NO: 1 - DNA, 516 bp, Homo sapiens
  source 1..516: mol_type = other DNA
atgcatcccttctacacccgggccgccaccatgataggcgagatcgccgccgccgtgtccttcatctccaagtttctccgcaccaaggggctcacgagcgagcgacagctgcagaccttcagccagagcctgcaggagctgctggcagaacattataaacatcactggttcccagaaaagccatgcaagggatcgggttaccgttgtattcgcatcaaccataaaatggatcctctgattggacaggcagcacagcggattggactgagcagtcaggagctgttcaggcttctcccaagtgaactcacactctgggttgacccctatgaagtgtcctacagaattggagaggatggctccatctgtgtgctgtatgaagcctcaccagcaggaggtagcactcaaaacagcaccaacgtgcaaatggtagacagccgaatcagctgtaaggaggaacttctcttgggcagaacgagcccttccaaaaactacaatatgatgactgtatcaggttaa

SEQ ID NO: 2 - DNA, 20 bp, synthetic construct
  source 1..20: mol_type = other DNA
ttctgtaggacacttcatag

SEQ ID NO: 3 - DNA, 20 bp, synthetic construct
  source 1..20: mol_type = other DNA
aatggatcctctgattggac

SEQ ID NO: 4 - DNA, 20 bp, synthetic construct
  source 1..20: mol_type = other DNA
gcagtactactgagtttttc

SEQ ID NO: 5 - DNA, 20 bp, synthetic construct
  source 1..20: mol_type = other DNA
gcctagtctcggtaagagtg

(19) United States
(12) Patent Application Publication
(10) Pub. No.: US 2024/0401146 A1
(43) Pub. Date: Dec. 5, 2024

(54) METHODS FOR PREDICTING AND IMPROVING THE EFFICACY OF MCL-1 INHIBITOR THERAPY

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE COTE D'AZUR, Nice (FR); CENTRE LEON BERARD, Lyon (FR); UNIVERSITÉ CLAUDE BERNARD - LYON 1, Villeurbanne (FR); HOSPICES CIVILS DE LYON, Lyon (FR)

(72) Inventors: Pierre SUJOBERT, Lyon - Cedex 07 (FR); Patrick AUBERGER, Nice (FR); Lorric DELAGE, Lyon - Cedex 07 (FR); Jean-Pierre ROUAULT, Lyon - Cedex 07 (FR); Arnaud JACQUEL, Nice Cedex 3 (FR)

(21) Appl. No.: 18/698,213

(22) PCT Filed: Oct. 5, 2022
(86) PCT No.: PCT/EP2022/077641

§ 371 Date: Apr. 3, 2024

(30) Foreign Application Priority Data
Oct. 6, 2021 (EP) 21306400.9

(51) Int. Cl. C12Q 1/6886

(52) U.S. Cl. CPC C12Q 1/6886; C12Q 2600/106; C12Q 2600/156

(57) ABSTRACT

Investigating the impact of BTG1 inactivation in lymphoma, the Inventors have demonstrated its role as a driver of lymphomagenesis in a murine model. They have also described the phenotypic consequences of BTG1 inactivation in human lymphoma cell lines. Especially, they have demonstrated that the inactivation of BTG1 is associated with an increased sensitivity to MCL-1 inhibition, which paves the way to the development of a personalized treatment for patients with BTG1 mutated lymphomas and cancers with a BTG1 inactivation. Accordingly, in a first aspect, the present invention relates to a method for predicting the response to a MCL-1 inhibitor treatment in a patient suffering from a cancer with a BTG1 inactivation, comprising the step of determining in a biological sample obtained from said patient the BTG1 mutation status, wherein a BTG1 inactivation is predictive of a response to a MCL-1 inhibitor treatment. In a second aspect, the present invention relates to a method of treating a patient suffering from a cancer with a BTG1 inactivation comprising the step of determining the BTG1 mutation status and administering a therapeutically effective amount of MCL-1 inhibitor when the patient carries a BTG1 gene inactivation.

### FIELD OF THE INVENTION

The present invention is in the field of medicine, in particular oncology, to predict and improving the efficacy of MCL-1 inhibitor therapies.

### BACKGROUND OF THE INVENTION

BTG1 is a highly conserved gene, which acts as a tumour suppressor gene in B-cell acute lymphoblastic leukaemia (J. P. Rouault et al., 1992; Waanders et al., 2012). The functions of this gene are poorly understood, except for its interactions with members of the mRNA de-adenylation complex (J.-P. Rouault et al., 1998), and with the arginine methyltransferase PRMT1 (Berthet et al., 2002; Lin, Gary, Yang, Clarke, & Herschman, 1996).

Diffuse large B cell lymphoma (DLBCL) is the most common subtype of lymphoma. Around two thirds of the patients can be cured with intensive chemotherapy, but this disease remains an unmet medical need for unfit patients or patients with relapse/refractory disease. In the recent years, genomic studies have defined molecular subgroups of clinical significance (Chapuy et al., 2018; Schmitz et al., 2018). Among them, the MCD/C5 subtype, characterized by MYD88 L265P and CD79B mutations is associated with extra nodal involvement, and very poor prognosis with immunochemotherapy. Interestingly, this subtype is especially enriched in mutations of BTG1 (40% of the patients). Many observations support the hypothesis of a driver role of BTG1 mutations: 1/most of the missense mutations tested have functional consequences on the capacity of BTG1 to interact with its partner CAF1 (Almasmoum, Airhihen, Seedhouse, & Winkler, 2020), 2/around 15% of mutations are truncating mutations predicted to have a strong impact on protein functions, 3/BTG1 mutations are highly enriched in post-germinal center derived lymphomas (DLBCL-ABC), and BTG1 deletions are highly prevalent in Waldenstrom macroglobulinemia (another lymphoma derived from post-GC B cells)(Hunter et al., 2014), and 5/BTG1 mutations confer a dismal prognosis in DLBCL (Reddy et al., 2017).

### SUMMARY OF THE INVENTION

In a first aspect, the present invention relates to a method for predicting the response to a MCL-1 inhibitor treatment in a patient suffering from a cancer with a BTG1 inactivation, comprising the step of determining in a biological sample obtained from said patient the BTG1 mutation status, wherein a BTG1 inactivation is predictive of a response to a MCL-1 inhibitor treatment.

In a second aspect, the present invention relates to a method of treating a patient suffering from a cancer with a BTG1 inactivation comprising the step of:
Determining in a biological sample obtained from said patient the BTG1 mutation status,
Administering a therapeutically effective amount of MCL-1 inhibitor when the patient carries a BTG1 gene inactivation.

### DETAILED DESCRIPTION OF THE INVENTION

Investigating the impact of BTG1 inactivation in lymphoma, the Inventors have demonstrated its role as a driver of lymphomagenesis in a murine model. They have also described the phenotypic consequences of BTG1 inactivation in human lymphoma cell lines. Especially, they have demonstrated that the inactivation of BTG1 is associated with an increased sensitivity to MCL-1 inhibition, which paves the way to the development of a personalized treatment for patients with BTG1 mutated lymphomas.

### Method for Predicting the Response to a MCL-1 Inhibitor Therapy

A first aspect of the invention relates to a method for predicting the response to a MCL-1 inhibitor treatment in a patient suffering from a cancer with a BTG1 inactivation, comprising the step of determining in a biological sample obtained from said patient the BTG1 mutation status, wherein a BTG1 inactivation is predictive of a response to a MCL-1 inhibitor treatment.

In some embodiment, the cancer with a BTG1 inactivation is selected in the group consisting B lymphoid hemopathy, cutaneous squamous cell carcinoma, pancreatic cancer, acral melanoma or hystiocytosis.

B lymphoid hemopathy (Chappuy et al. 2018; Morin et al. 2011; Reddy et al. 2017; Braggio et al. 2015), cutaneous squamous cell carcinoma (Pickering et al. 2014), pancreatic cancer (Witkiewicz et al. 2015), acral melanoma (Liang et al. 2017) and hystiocytosis (Diamond et al. 2019) were demonstrated as particularly enriched in BTG1 mutations or deletions, leading to a BTG1 inactivation (FIG. 1).

As used herein, the term “patient” denotes a mammal, such as a rodent, a feline, a canine or a primate. Preferably, a patient according to the invention is a human. Typically, a patient according to the invention refers to any subject (preferably human) afflicted with or susceptible to be afflicted with a cancer with a BTG1 inactivation, in particular a B cell lymphoma, in particular a Diffuse Large B Cell Lymphoma (DLBCL).

As used herein, the term “biological sample” refers to any biological sample of a subject and can include, by way of example and not limitation, bodily fluids and/or tissue extracts such as homogenates or solubilized tissue obtained from a subject. Tissue extracts are obtained routinely from tissue biopsy. As example, a biological sample may be a tumor sample, bodily fluids such as blood sample, plasma sample, serum sample, cerebrospinal fluid, pleural effusion, ascetic effusion, urinary sample, saliva sample, or in case of fetus, amniotic fluid or chorionic villy or any other bodily secretion or derivative thereof. In some embodiment, the biological sample is a tumor sample or a bodily fluid sample which may contain cell free DNA/RNA associated tumour (circulating tumour DNA/RNA). In some embodiment, when the patient suffers from B-cell lymphoma, the sample is a lymphatic tissue, bone, cerebrospinal fluid, digestive tract, sinus, testicles, thyroid gland or skin. In a more particular embodiment, the biological sample is bodily fluid selected from the list consisting of cerebrospinal fluid, pleural effusion, ascitic effusion, plasma, serum, urine which may contain cell free DNA/RNA associated tumour (circulating tumour DNA/RNA). According to the invention, a BTG1 inactivation is detected by using any type of tumour cell or cell free DNA/RNA (circulating tumour DNA/RNA).

As used herein, the term “circulating tumour DNA/RNA” or “ctDNA” refers to tumor-derived fragmented DNA in the bloodstream that is not associated with cells. Because ctDNA may reflect the entire tumor genome, it has gained traction for its potential clinical utility; “bodily fluid biopsies” in the form of blood draws may be taken at various time points to monitor tumor progression throughout the treatment regimen (Wan J, et al (2017). “Nature Reviews Cancer. 17 (4): 223-238).

As used herein, the term “response to a MCL-1 inhibitor treatment” refers to a clinically significant relief in the disease when treated with a MCL-1 inhibitor.

As used herein, the term “B lymphoid hemopathy” denotes a disease resulting from the neoplastic transformation of a B cell, impacting immature or mature B-cells, leading respectively to B lymphoblastic leukemia or to mature B-cell lymphoma. Thus, in some embodiment, the B lymphoid hemopathy is a B lymphoblastic leukemia or a mature B-cell lymphoma.

As used herein, the term “B lymphoblastic leukemia” is a cancer of the lymphoid line of blood cells developed in bone marrow and characterized by the neoplastic transformation of immature lymphocytes. Normal lymphoblasts develop into mature infection-fighting B-cells (lymphocytes). In lymphoblastic leukemia, both normal development of some lymphocytes and the control over the number of lymphoid cells become defective (Terwilliger T et al. Acute lymphoblastic leukemia: a comprehensive review and 2017 update. Blood Cancer J. 2017; 7(6):e577. Published 2017 Jun. 30. doi:10.1038/bcj.2017.53).

As used herein, the term “lymphoma” refers to a group of blood cell tumours that develop from lymphoid cells. The two main categories of lymphomas are Hodgkin lymphomas (HL) and non-Hodgkin lymphomas (NHL). NHL lymphomas are categorized by affected cell type: B-cell lymphoma or T-cell lymphoma.

“Mature B-cell lymphoma” comprises different subtypes: Diffuse Large B-cell Lymphoma (DLBCL), follicular lymphoma, Waldenstrom macroglobulinemia, mantle cell lymphoma, marginal zone B-cell lymphoma, Burkitt lymphoma, hairy cell leukemia and chronic lymphocytic leukemia among others (see WHO 2016).

In some embodiment, the mature B-Cell lymphoma is a Diffuse Large B Cell Lymphoma.

As used herein, the term “DLBCL” or “Diffuse Large B Cell Lymphoma” is a non-Hodgkin lymphoma developed from B lymphocytes, where B cells are abnormally larger than normal and healthy B cells. DLBCL is the most common form of adult lymphoma worldwide. As example, a DLBCL may be diagnosed with an excisional biopsy of an abnormal lymph node, the excisional biopsy showing a disrupted structural integrity of the lymph node architecture with large cells. A DLBCL may be an activated B Cell Derived Lymphoma (“ABC”) subtype or a Germinal Center Derived Lymphoma (“GC”) subtype (Alizadeh, A., et al. Distinct types of diffuse large B-cell lymphoma identified by gene expression profiling. Nature 403, 503-511 (2000). https://doi.org/10.1038/35000501), according to Cell-Of-Origin (COO).

As example, a DLBCL subtype may be classified with a Reverse-Transcriptase Multiplex Ligation-dependent Probe Amplification Classifier (RT-MLPA; Bobée V. et al. Determination of Molecular Subtypes of Diffuse Large B-Cell Lymphoma Using a Reverse Transcriptase Multiplex Ligation-Dependent Probe Amplification Classifier: A CALYM Study. J Mol Diagn. 2017; 19(6):892-904. doi:10.1016/j.jmoldx.2017.07.007) or a nanostring Lymphoma Subtyping Test (LST; Michaelsen T Y et al. A B-cell-associated gene signature classification of diffuse large B-cell lymphoma by NanoString technology. Blood Adv. 2018; 2(13):1542-1546. doi:10.1182/bloodadvances.2018017988).

In some embodiment, the DLBCL is a Germinal Center Derived Lymphoma (“GC”) subtype. A GC DLBCL subtype is characterized by B cells arising from normal germinal center. As example, a GC B-cell subtype phenotype may be CD10⁺ or CD10⁻BCL6⁺MUM1⁻.

In some embodiment, the DLBCL is an Activated B-cell (“ABC”, also known as “Post Germinal Center Derived Lymphoma”) lymphoma subtype. An ABC subtype is characterized by B cells arising after the germinal center reaction. As example, an ABC subtype phenotype may be CD10⁻BCL6⁻ or CD10⁻BCL⁺MUM1⁺.

In some embodiment, the DLBCL subtype is a MCD/C5 subtype. A MCD/C5 subtype is characterized by MYD88 (Gene ID:4615) p.L265P and CD79B (Gene ID:974) gene mutation. MYD88 gene is involved in the regulation of NF-KB and MAPK-ERK signaling pathway that control cell proliferation and survival. CD79B gene is involved in the signal transduction of the B cell receptor (BCR). MCD/C5 subtype are predominantly (96%) ABC subtype (Schmitz R. et al. Genetics and Pathogenesis of Diffuse Large B-Cell Lymphoma. N Engl J Med. 2018; 378(15):1396-1407. doi:10.1056/NEJMoa1801445).

As used herein, the term “cutaneous squamous cell carcinoma” denotes the second most common of skin cancer, characterized by abnormal, accelerated growth of squamous cells.

As used herein, the term “pancreatic cancer” denotes a pathological condition in mammals that is typically characterized by unregulated pancreas cell growth. More precisely, pancreatic cancer is a malignant tumor characterized by rapid progression that affects the exocrine compartment.

As used herein, the term “acral melanoma” denotes a type of melanoma arising on the palm, sole or beneath the nail, from pigment cells (melanocytes).

As used herein, the term “hystiocytosis” denotes a generic name for a group of syndromes characterized by an abnormal increase in the number of histiocytes.

As used herein, the term “BTG1” or “B cell Translocation Gene 1” denotes a member of the TOB/BTG family of proteins, known to inhibits cell proliferation and negatively regulate the cell cycle (SEQ ID NO:1, Gene ID: 694; Ensembl: ENSG00000133639; Uniprot: P62324) (Ikematsu N, et al. Tob2, a novel anti-proliferative Tob/BTG1 family member, associates with a component of the CCR4 transcriptional regulatory complex capable of binding cyclin-dependent kinases. Oncogene. 1999; 18(52):7432-7441. doi:10.1038/sj.onc.1203193). BTG1 protein is localized in nucleus and cytoplasm and can interact with CAF1 (Rouault et al., 1998) and PRMT1 (Liu C. et al. BTG1 potentiates apoptosis and suppresses proliferation in renal cell carcinoma by interacting with PRMT1. Oncol Lett. 2015; 10(2):619-624. doi:10.3892/ol.2015.3293).

As used herein, the term “BTG1 inactivation” denotes a state wherein BTG1 gene is mutated and/or deleted, or when its expression is repressed either at the transcriptional level or at the post-transcriptional level. In particular, a BTG1 inactivation lead to at least one mutation that totally or partially impairs BTG1 gene expression and/or BTG1 protein activity either because the protein is lacking (e.g. deleted gene, inhibited expression), or is defective (e.g. mutated gene). Such mutation may be a missense mutation (i.e. a single nucleotide change results in a codon that encodes for a different amino acid), a nonsense mutation (i.e. a mutation that results in a premature stop codon), a deletion and/or an insertion (i.e. respectively the suppression or the addition of at least one nucleotide). As example, a BTG1 inactivation may be established by sequencing genomic DNA or by sequencing ctDNA, or sequencing or assaying BTG1 mRNA expression in a biological sample. If a BTG1 inactivation is characterized, the patient is said to be a carrier of a BTG1 inactivation.

According to the invention, the determination of the presence or absence of said BTG1 inactivation may be determined by DNA sequencing, PCR analysis or any genotyping method known in the art. Examples of such methods include, but are not limited to, chemical assays such as allele specific hybridation, primer extension, allele specific oligonucleotide ligation, sequencing, enzymatic cleavage, flap endonuclease discrimination; and detection methods such as fluorescence, chemiluminescence, and mass spectrometry.

For example, the presence or absence of said inactivation may be detected in a DNA sample, preferably after amplification. For instance, the isolated DNA may be subjected to amplification by polymerase chain reaction (PCR), using specific oligonucleotide primers that are specific for the BTG1 inactivation or that enable amplification of a flanking region. According to a first alternative, conditions for primer annealing may be chosen to ensure specific amplification; so that the appearance of an amplification product be a diagnostic of the presence of the BTG1 inactivation according to the invention. Otherwise, DNA may be amplified, after which a mutated site may be detected in the amplified sequence by hybridization with a suitable probe or by direct sequencing, or any other appropriate method known in the art.

Actually numerous strategies for genotype analysis are available (Cooper et al., 1991; Grompe, 1993). Briefly, the nucleic acid molecule may be tested for the presence or absence of a restriction site. When a base polymorphism creates or abolishes the recognition site of a restriction enzyme, this allows a simple direct PCR genotype of the polymorphism. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; Next-generation sequencing technologies including targeted-NGS panel, exome and whole genome sequencing methods; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the Invader™ assay, or oligonucleotide ligation assay (OLA). OLA may be used for revealing base polymorphisms. According to this method, two oligonucleotides are constructed that hybridize to adjacent sequences in the target nucleic acid, with the join sited at the position of the polymorphism. DNA ligase will covalently join the two oligonucleotides only if they are perfectly hybridized to one of the allele.

Therefore, short DNA sequences, in particular oligonucleotide probes or primers, according to the present invention include those which specifically hybridize the one of the allele of the polymorphism.

Oligonucleotide probes or primers may contain at least 10, 15, 20 or 30 nucleotides. Their length may be shorter than 400, 300, 200 or 100 nucleotides.

According to the invention, the determination of the presence or absence of said BTG1 inactivation may also be determined by detection or not of the mutated BTG1 protein(s) (i.e. BTG1 protein mature form (without signal peptide)) by any method known in the art. The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal. As used herein, the term “labelled” with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as I123, I124, In111, Re186 and Re188. The aforementioned assays generally involve separation of unbound protein in a liquid phase from a solid phase support to which antigen-antibody complexes are bound. Solid supports which may be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, etc.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used. IHC specifically provides a method of detecting a target in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, AZ), the NBA™ kit (Zymed Laboratories Inc., South San Francisco, CA), HistoFine® (Nichirei Corp, Tokyo, Japan).

In particular embodiment, a tissue section (e.g. a tissue sample or biopsy) may be mounted on a slide or other support after incubation with antibodies directed against the BTG1 protein. Then, microscopic inspections in the sample mounted on a suitable solid support may be performed. For the production of photomicrographs, sections comprising samples may be mounted on a glass slide or other planar support, to highlight by selective staining the presence of the protein of interest. Therefore IHC samples may include, for instance: (a) preparations comprising cell samples (b) fixed and embedded said cells and (c) detecting the protein of interest in said cell samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

In some embodiment, the BTG1 inactivation results from a mutation selected in the group consisting in 12:g.92537857T>G, 12:g.92537876T>A, 12:g.92537878T>C, 12:g.92537886T>G, 12:g.92537888T>A, 12:g.92537899G>A, 12:g.92537900T>C, 12:g.92537926C>T, 12:g.92537942C>A, 12:g.92537942C>G, 12:g.92537945C>T, 12:g.92537960T>A, 12:g.92537964_92537965insTT, 12:g.92537974C>T, 12:g.92537984C>T, 12:g.92537992G>A, 12:g.92537996C>T, 12:g.92537998T>C, 12:g.92538010A>G, 12:g.92538021C>A, 12:g.92538029T>A, 12:g.92538029T>C, 12:g.92538031C>A, 12:g.92538031C>T, 12:g.92538040A>T, 12:g.92538041C>T, 12:g.92538056C>T, 12:g.92538061A>T, 12:g.92538068G>T, 12:g.92538071C>T, 12:g.92538092G>C, 12:g.92538121G>A, 12:g.92538121G>T, 12:g.92538122C>T, 12:g.92538125C>T, 12:g.92538131C>T, 12:g.92538147T>G, 12:g.92538163A>G, 12:g.92538164T>C, 12:g.92538164T>G, 12:g.92538165A>C, 12:g.92538166C>T, 12:g.92538169C>A, 12:g.92538169C>T, 12:g.92538171G>C, 12:g.92538173A>G, 12:g.92538175C>A, 12:g.92538175C>T, 12:g.92538176C>G, 12:g.92538181C>T, 12:g.92538182C>T, 12:g.92538187C>A, 12:g.92538187C>T, 12:g.92538191G>A, 12:g.92538195T>A, 12:g.92538204C>G, 12:g.92538204C>T, 12:g.92538205C>T, 12:g.92538208T>C, 12:g.92538209G>A, 12:g.92538212G>A, 12:g.92538214T>C, 12:g.92538220T>G, 12:g.92538230_92538234del, 12:g.92539158G>A, 12:g.92539159C>A, 12:g.92539159C>T, 12:g.92539163C>A, 12:g.92539164C>T, 12:g.92539167C>T, 12:g.92539172A>T, 12:g.92539173G>C, 12:g.92539174_92539180del, 12:g.92539174C>A, 12:g.92539174C>G, 12:g.92539175T>A, 12:g.92539176C>G, 12:g.92539176C>T, 12:g.92539178T>G, 12:g.92539179G>A, 12:g.92539184C>T, 12:g.92539193A>C, 12:g.92539196G>A, 12:g.92539200G>A, 12:g.92539200G>C, 12:g.92539203G>C, 12:g.92539203G>T, 12:g.92539204C>G, 12:g.92539209G>A, 12:g.92539209G>C, 12:g.92539210C>G, 12:g.92539214C>T, 12:g.92539221G>A, 12:g.92539223C>T, 12:g.92539224C>T, 12:g.92539227T>A, 12:g.92539227T>G, 12:g.92539229G>C, 12:g.92539232C>T, 12:g.92539235A>G, 12:g.92539238A>C, 12:g.92539239A>T, 12:g.92539241T>C, 12:g.92539245A>C, 12:g.92539246G>C, 12:g.92539248T>C, 12:g.92539248T>G, 12:g.92539251A>G, 12:g.92539257C>T, 12:g.92539259G>A, 12:g.92539260C>T, 12:g.92539262G>A, 12:g.92539270C>G, 12:g.92539278T>G, 12:g.92539279C>T, 12:g.92539283G>A, 12:g.92539289G>T, 12:g.92539299A>G, 12:g.92539304_92539305delinsAC, 12:g.92539304G>A, 12:g.92539304G>C, 12:g.92539304G>T, 12:g.92539308G>A, 12:g.92539308G>T, 12:g.92539309C>T.

In some embodiment, the BTG1 inactivation results from a missense mutation selected in the group consisting in 12:g.92537876T>A, 12:g.92537878T>C, 12:g.92537886T>G, 12:g.92537899G>A, 12:g.92537900T>C, 12:g.92537926C>T, 12:g.92537942C>A, 12:g.92537942C>G, 12:g.92537945C>T, 12:g.92537960T>A, 12:g.92537974C>T, 12:g.92537984C>T, 12:g.92537992G>A, 12:g.92537996C>T, 12:g.92537998T>C, 12:g.92538010A>G, 12:g.92538021C>A, 12:g.92538029T>A, 12:g.92538029T>C, 12:g.92538031C>A, 12:g.92538031C>T, 12:g.92538040A>T, 12:g.92538041C>T, 12:g.92538056C>T, 12:g.92538061A>T, 12:g.92538068G>T, 12:g.92538071C>T, 12:g.92538092G>C, 12:g.92538121G>A, 12:g.92538121G>T, 12:g.92538122C>T, 12:g.92538125C>T, 12:g.92538131C>T, 12:g.92538147T>G, 12:g.92538163A>G, 12:g.92538164T>C, 12:g.92538164T>G, 12:g.92538165A>C, 12:g.92538166C>T, 12:g.92538169C>A, 12:g.92538169C>T, 12:g.92538173A>G, 12:g.92538175C>A, 12:g.92538175C>T, 12:g.92538176C>G, 12:g.92538181C>T, 12:g.92538182C>T, 12:g.92538187C>A, 12:g.92538187C>T, 12:g.92538191G>A, 12:g.92538195T>A, 12:g.92538204C>G, 12:g.92538208T>C, 12:g.92538209G>A, 12:g.92538212G>A, 12:g.92538214T>C, 12:g.92538220T>G, 12:g.92539164C>T, 12:g.92539167C>T, 12:g.92539172A>T, 12:g.92539173G>C, 12:g.92539174C>A, 12:g.92539174C>G, 12:g.92539175T>A, 12:g.92539176C>G, 12:g.92539176C>T, 12:g.92539178T>G, 12:g.92539184C>T, 12:g.92539193A>C, 12:g.92539196G>A, 12:g.92539200G>C, 12:g.92539203G>C, 12:g.92539203G>T, 12:g.92539204C>G, 12:g.92539209G>C, 12:g.92539210C>G, 12:g.92539214C>T, 12:g.92539221G>A, 12:g.92539223C>T, 12:g.92539224C>T, 12:g.92539227T>G, 12:g.92539229G>C, 12:g.92539232C>T, 12:g.92539235A>G, 12:g.92539238A>C, 12:g.92539239A>T, 12:g.92539241T>C, 12:g.92539245A>C, 12:g.92539246G>C, 12:g.92539248T>C, 12:g.92539248T>G, 12:g.92539251A>G, 12:g.92539257C>T, 12:g.92539259G>A, 12:g.92539260C>T, 12:g.92539262G>A, 12:g.92539270C>G, 12:g.92539278T>G, 12:g.92539279C>T, 12:g.92539283G>A, 12:g.92539289G>T, 12:g.92539299A>G, 12:g.92539304_92539305delinsAC, 12:g.92539304G>A, 12:g.92539304G>C, 12:g.92539304G>T, 12:g.92539308G>A, 12:g.92539308G>T

In some embodiment, the BTG1 inactivation results from a missense mutation selected in the group consisting in L26P, G66D, I115V, A127V, A130T, A17V, A18T, A18V, A49T, A83T, A84E, A84T, A84V, A8D, C149Y, C62F, C62Y, C69W, C69Y, D144H, D144Y, E101K, E117D, E126K, E14D, E34D, E46D, E46K, E46Q, E46V, E50K, E59D, F21L, F25C, F25I, F40C, G30E, G30R, G64E, G64R, G66R, G66V, G81R, H2N, H2Y, H51P, H54Y, H55R, H55Y, I115F, I121T, I12L, I22L, I22M, I22V, I70L, I70T, I70V, K162N, K24R, K29Q, K53R, K75N, L1021, L104H, L31F, L37M, L37V, L47Q, L47V, L94V, M11I, M166L, N165S, P3H, P3L, P3R, P3V, P61S, Q36H, Q38E, Q45P, R1141, R114K, R27H, R35G, R68H, R68L, S133N, S23A, S33N, S43N, T10I, T138S, T158A, T158M, T28S, T391, V106I, V111E, V111M, V143I, V19M, W56C, Y125C, Y5H, Y67H.

In some embodiment, the BTG1 inactivation results from a missense mutation selected in the group consisting in L26P, G66D and/or I115V.

In some embodiment, the BTG1 inactivation impairs BTG1 interaction with CAF1 or PRMT1. The skilled person well-know how to identify an impaired BTG1 interaction with another protein, as example with a co-immunoprecipitation test (Lee C. Coimmunoprecipitation assay. Methods Mol Biol. 2007; 362:401-406. doi:10.1007/978-1-59745-257-1_31), yeast two-hybrid and bacterial two-hybrid screen (Battesti A, Bouveret E. The bacterial two-hybrid system based on adenylate cyclase reconstitution in Escherichia coli. Methods. 2012; 58(4):325-334. doi:10.1016/j.ymeth.2012.07.018), affinity electrophoresis (Aizpurua-Olaizola O, Sastre Torano J, Pukin A, et al. Affinity capillary electrophoresis for the assessment of binding affinity of carbohydrate-based cholera toxin inhibitors. Electrophoresis. 2018; 39(2):344-347. doi:10.1002/elps.201700207), phage display (Smith G P, Petrenko V A. Phage Display. Chem Rev. 1997; 97(2):391-410. doi:10.1021/cr960065d), fluorescence resonance affinity transfer (Martin S F, Tatham M H, Hay R T, Samuel I D. Quantitative analysis of multi-protein interactions using FRET: application to the SUMO pathway. Protein Sci. 2008; 17(4):777-784. doi:10.1110/ps.073369608) or chemical cross-linking.

As used herein, the term “CAF1” or “cNOT7” denotes a protein encoded by cNOT7 gene (Gene ID: 29883; Ensembl: ENSG000000198791; Uniprot: Q9UIV1). The protein encoded by this gene binds to a B-cell translocation protein 1, which negatively regulates cell proliferation. Binding of the two proteins, which is driven by phosphorylation of the anti-proliferative protein, causes signaling events in cell division that lead to changes in cell proliferation associated with cell-cell contact.

As used herein, the term “PRMT1” or “Protein Arginine MethylTransferase 1” denotes a protein member of the protein arginine N-methyltransferase (PRMT) family. Post-translational modification of target proteins by PRMTs plays an important regulatory role in many biological processes, whereby PRMTs methylate arginine residues by transferring methyl groups from S-adenosyl-L-methionine to terminal guanidino nitrogen atoms. The encoded protein is a type I PRMT and is responsible for the majority of cellular arginine methylation activity. PRMT1 is encoded by PRMT1 gene (Gene ID: 3276; Ensembl: ENSG00000126457; UniProt: Q99873).

As used herein, the term “MCL-1” or “Myeloid Cell Leukemia 1” denotes an anti-apoptotic protein of the Bcl-2 family encoded by the MCL-1 gene (Gene ID:4170; Ensembl: ENSG00000143384; UniProt: Q07820). MCL-1 is involved in the regulation of apoptosis, and in the maintenance of viability but not of proliferation. MCL-1 can be cleaved by caspases during apoptosis to produce a cell death promoting molecule. This protein is rapidly degraded in response to cell death signals and immediately re-induced by survival stimuli. This protein mediates its effects by interactions with a number of other regulators such as BAKI, BAX, BCL2L11, BID, BAD, DAD1, PMAIP1, PCNA, TCTP and/or TNKS.

As used herein, the term “MCL-1 inhibitor” denotes a molecule or compound which can inhibit directly or indirectly the activity of the protein by limiting or impairing the interactions of the protein, or a molecule or compound which destabilizes the protein structure, or a molecule or compound which inhibits the transcription or the translation of MCL1, or accelerates its degradation. The term “MCL-1 inhibitor” also denotes an inhibitor of the expression of the gene coding for the protein. As example, MCL-1 inhibitors demonstrate a pro-apoptotic activity by targeting transcriptional regulators of MCL-1, BH3 mimetic disruption, promoting ubiquitination of MCL-1 or targeting deubiquitin enzymes upstream of MCL-1 (Wu X. Ubiquitination and deubiquitination of MCL-1 in cancer: deciphering chemoresistance mechanisms and providing potential therapeutic options. Cell Death Dis. 2020; 11(7):556. Published 2020 Jul. 22. doi:10.1038/s41419-020-02760-y). Others examples of indirect MCL-1 inhibitors may be mTORC1 inhibitors (Li, H, Liu, L., Chang, H. et al. Downregulation of MCL-1 and upregulation of PUMA using mTOR inhibitors enhance antitumor efficacy of BH3 mimetics in triple-negative breast cancer. Cell Death Dis 9, 137 (2018). https://doi.org/10.1038/s41419-017-0169-2) or biguanides (Yue W, Zheng X, Lin Y, et al. Metformin combined with aspirin significantly inhibit pancreatic cancer cell growth in vitro and in vivo by suppressing anti-apoptotic proteins Mcl-1 and Bcl-2. Oncotarget. 2015; 6(25):21208-21224. doi:10.18632/oncotarget.4126).

MCL-1 inhibitors are well known in the art. Examples of patents disclosing MCL-1 inhibitors are WO2017/182625, WO2018/183418, WO2011/094708, WO2015/153959, WO2013/138668 or WO2016/207225.

The following MCL-1 inhibitors are commercially available: S63845 (Servier, Suresnes, FR; CAS No. 1799633-27-4), MIK665 (Novartis, Bale, CH; CAS No. 1799631-75-6; NCT02992483), ABBV-467 (AbbVie, North Chicago, USA. CAS No. 2138861-99-9; NCT04178902), S64315 (Servier, Suresnes, FR; CAS No. 1799631-75-6; NCT02979366 and NCT03672695), PRT1419 (Prelude Therapeutics, Wilmington, DE; CAS No. 31083-55-3; NCT04543305), AMG397 (Amgen, Thousand Oaks, USA; CAS No. 2245848; NCT03465540), AMG176 (Amgen, Thousand Oaks, USA; CAS No. 1883727-34-1; NCT02675452), AZD5991 (AstraZeneca, Londres, EN; CAS No. 2143010-83-5; NCT03218683).

Other example of MCL-1 inhibitors are marinopyrrole A (CAS No. 1227962-62-0), VU661013 (CAS No. 2131184-57-9), A-1210477 (CAS No. 1668553-26-1), BL-08040 (CAS No. 664334-36-5), omacetaxine mepesuccinate (CAS No. 26833-87-4), UMI77 (CAS No. 518303-20-3) or CYC202 (CAS No. 186692-46-6).

Thus, in some embodiment, the MCL-1 inhibitor is S63845, MIK665, ABBV-467, 564315, PRT1419, AMG397, AMG176 and/or AZD5991.

In some embodiment, the MCL-1 inhibitor is marinopyrrole A, VU661013, A-1210477, BL-08040, omacetaxine mepesuccinate, UM177 and/or CYC202.

In some embodiment, the MCL-1 inhibitor according to the invention may be a low molecular weight compound, e.g. a small organic molecule (natural or not). The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In some embodiment, the MCL-1 inhibitor according to the invention is an antibody. Antibodies directed against MCL-1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against MCL-1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-MCL-1 single chain antibodies. Compounds useful in practicing the present invention also include anti-MCL-1 antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to MCL-1. Humanized anti-MCL-1 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In the context of the invention, it could be advantageous to use a nanobody directed against MCL-1 in order to enter the cell. Thus in another embodiment, the antibody according to the invention is a single domain antibody directed against MCL-1. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH. The VHH according to the invention can readily be prepared by an ordinarily skilled artisan using routine experimentation. The VHH variants and modified form thereof may be produced under any known technique in the art such as in-vitro maturation. VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

In one embodiment, the compound according to the invention is a polypeptide. In a particular embodiment the polypeptide is an inhibitor of MCL-1 and is capable to prevent the function of MCL-1. Particularly, the polypeptide can be a mutated MCL-1 protein or a similar protein without the function of MCL-1. In the context of the invention, it could be advantageous to use a polypeptide inhibitor of MCL-1 linked to a cell-penetrating peptide in order to enter the cell. Thus in one embodiment, the polypeptide of the invention may be linked to a cell-penetrating peptide” to allow the penetration of the polypeptide in the cell. The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012). The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli. In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain. Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the MCL-1 inhibitor according to the invention is an inhibitor of MCL-1 gene expression.

Small inhibitory RNAs (siRNAs) can also function as inhibitors of MCL-1 expression for use in the present invention. MCL-1 gene expression can be reduced by contacting a subject or cell with a small double stranded RNA (dsRNA), or a vector or construct causing the production of a small double stranded RNA, such that MCL-1 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). As example, siRNAs directed against MCL-1 are described in Li B P et al. (“Effects of siRNA-mediated silencing of myeloid cell leukelia-1 on the biological behaviors and drug resistance of gastric cancer cells”. Am J Transl Res. 2015; 7(11):2397-2411. Published 2015 Nov. 15): Mcl-1-siRNA-892, Mcl-1-siRNA-927, Mcl-1-siRNA-1023 and Mcl-1-siRNA-1543.

Ribozymes can also function as inhibitors of MCL-1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of MCL-1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of MCL-1 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing MCL-1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991. Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUCl9, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter can also be, e.g., a viral promoter, such as CMV promoter or any synthetic promoters.

### Therapeutic Method

A second aspect of the present invention relates to a method of treating a patient suffering from a cancer with a BTG1 inactivation comprising the step of:
Determining in a biological sample obtained from said patient the BTG1 mutation status,
Administering a therapeutically effective amount of MCL-1 inhibitor when the patient carries a BTG1 gene inactivation.

In some embodiment, the cancer with a BTG1 inactivation is selected in the group consisting B lymphoid hemopathy, cutaneous squamous cell carcinoma, pancreatic cancer, acral melanoma or hystiocytosis.

In some embodiment, B lymphoid hemopathy is a B lymphoblastic leukemia or a mature B-cell lymphoma.

In some embodiment, the mature B-Cell lymphoma is a diffuse large B-cell lymphoma (DLBCL), follicular lymphoma, Waldenstrom macroglobulinemia, mantle cell lymphoma, marginal zone B-cell lymphoma, Burkitt lymphoma, hairy cell leukemia or chronic lymphocytic leukemia.

In some embodiment, the B-Cell lymphoma is a Diffuse Large B Cell Lymphoma (DLBCL).

In some embodiment, the DLBCL is a Germinal Center Derived Lymphoma subtype.

In some embodiment, the DLBCL is an Activated B-Cell lymphoma subtype.

In some embodiment, the DLBCL is a MCD/C5 subtype.

In some embodiment, the BTG1 inactivation results from a mutation selected in the group consisting in 12:g.92537857T>G, 12:g.92537876T>A, 12:g.92537878T>C, 12:g.92537886T>G, 12:g.92537888T>A, 12:g.92537899G>A, 12:g.92537900T>C, 12:g.92537926C>T, 12:g.92537942C>A, 12:g.92537942C>G, 12:g.92537945C>T, 12:g.92537960T>A, 12:g.92537964_92537965insTT, 12:g.92537974C>T, 12:g.92537984C>T, 12:g.92537992G>A, 12:g.92537996C>T, 12:g.92537998T>C, 12:g.92538010A>G, 12:g.92538021C>A, 12:g.92538029T>A, 12:g.92538029T>C, 12:g.92538031C>A, 12:g.92538031C>T, 12:g.92538040A>T, 12:g.92538041C>T, 12:g.92538056C>T, 12:g.92538061A>T, 12:g.92538068G>T, 12:g.92538071C>T, 12:g.92538092G>C, 12:g.92538121G>A, 12:g.92538121G>T, 12:g.92538122C>T, 12:g.92538125C>T, 12:g.92538131C>T, 12:g.92538147T>G, 12:g.92538163A>G, 12:g.92538164T>C, 12:g.92538164T>G, 12:g.92538165A>C, 12:g.92538166C>T, 12:g.92538169C>A, 12:g.92538169C>T, 12:g.92538171G>C, 12:g.92538173A>G, 12:g.92538175C>A, 12:g.92538175C>T, 12:g.92538176C>G, 12:g.92538181C>T, 12:g.92538182C>T, 12:g.92538187C>A, 12:g.92538187C>T, 12:g.92538191G>A, 12:g.92538195T>A, 12:g.92538204C>G, 12:g.92538204C>T, 12:g.92538205C>T, 12:g.92538208T>C, 12:g.92538209G>A, 12:g.92538212G>A, 12:g.92538214T>C, 12:g.92538220T>G, 12:g.92538230_92538234del, 12:g.92539158G>A, 12:g.92539159C>A, 12:g.92539159C>T, 12:g.92539163C>A, 12:g.92539164C>T, 12:g.92539167C>T, 12:g.92539172A>T, 12:g.92539173G>C, 12:g.92539174_92539180del, 12:g.92539174C>A, 12:g.92539174C>G, 12:g.92539175T>A, 12:g.92539176C>G, 12:g.92539176C>T, 12:g.92539178T>G, 12:g.92539179G>A, 12:g.92539184C>T, 12:g.92539193A>C, 12:g.92539196G>A, 12:g.92539200G>A, 12:g.92539200G>C, 12:g.92539203G>C, 12:g.92539203G>T, 12:g.92539204C>G, 12:g.92539209G>A, 12:g.92539209G>C, 12:g.92539210C>G, 12:g.92539214C>T, 12:g.92539221G>A, 12:g.92539223C>T, 12:g.92539224C>T, 12:g.92539227T>A, 12:g.92539227T>G, 12:g.92539229G>C, 12:g.92539232C>T, 12:g.92539235A>G, 12:g.92539238A>C, 12:g.92539239A>T, 12:g.92539241T>C, 12:g.92539245A>C, 12:g.92539246G>C, 12:g.92539248T>C, 12:g.92539248T>G, 12:g.92539251A>G, 12:g.92539257C>T, 12:g.92539259G>A, 12:g.92539260C>T, 12:g.92539262G>A, 12:g.92539270C>G, 12:g.92539278T>G, 12:g.92539279C>T, 12:g.92539283G>A, 12:g.92539289G>T, 12:g.92539299A>G, 12:g.92539304_92539305delinsAC, 12:g.92539304G>A, 12:g.92539304G>C, 12:g.92539304G>T, 12:g.92539308G>A, 12:g.92539308G>T, 12:g.92539309C>T.

In some embodiment, the BTG1 inactivation results from a missense mutation selected in the group consisting in 12:g.92537876T>A, 12:g.92537878T>C, 12:g.92537886T>G, 12:g.92537899G>A, 12:g.92537900T>C, 12:g.92537926C>T, 12:g.92537942C>A, 12:g.92537942C>G, 12:g.92537945C>T, 12:g.92537960T>A, 12:g.92537974C>T, 12:g.92537984C>T, 12:g.92537992G>A, 12:g.92537996C>T, 12:g.92537998T>C, 12:g.92538010A>G, 12:g.92538021C>A, 12:g.92538029T>A, 12:g.92538029T>C, 12:g.92538031C>A, 12:g.92538031C>T, 12:g.92538040A>T, 12:g.92538041C>T, 12:g.92538056C>T, 12:g.92538061A>T, 12:g.92538068G>T, 12:g.92538071C>T, 12:g.92538092G>C, 12:g.92538121G>A, 12:g.92538121G>T, 12:g.92538122C>T, 12:g.92538125C>T, 12:g.92538131C>T, 12:g.92538147T>G, 12:g.92538163A>G, 12:g.92538164T>C, 12:g.92538164T>G, 12:g.92538165A>C, 12:g.92538166C>T, 12:g.92538169C>A, 12:g.92538169C>T, 12:g.92538173A>G, 12:g.92538175C>A, 12:g.92538175C>T, 12:g.92538176C>G, 12:g.92538181C>T, 12:g.92538182C>T, 12:g.92538187C>A, 12:g.92538187C>T, 12:g.92538191G>A, 12:g.92538195T>A, 12:g.92538204C>G, 12:g.92538208T>C, 12:g.92538209G>A, 12:g.92538212G>A, 12:g.92538214T>C, 12:g.92538220T>G, 12:g.92539164C>T, 12:g.92539167C>T, 12:g.92539172A>T, 12:g.92539173G>C, 12:g.92539174C>A, 12:g.92539174C>G, 12:g.92539175T>A, 12:g.92539176C>G, 12:g.92539176C>T, 12:g.92539178T>G, 12:g.92539184C>T, 12:g.92539193A>C, 12:g.92539196G>A, 12:g.92539200G>C, 12:g.92539203G>C, 12:g.92539203G>T, 12:g.92539204C>G, 12:g.92539209G>C, 12:g.92539210C>G, 12:g.92539214C>T, 12:g.92539221G>A, 12:g.92539223C>T, 12:g.92539224C>T, 12:g.92539227T>G, 12:g.92539229G>C, 12:g.92539232C>T, 12:g.92539235A>G, 12:g.92539238A>C, 12:g.92539239A>T, 12:g.92539241T>C, 12:g.92539245A>C, 12:g.92539246G>C, 12:g.92539248T>C, 12:g.92539248T>G, 12:g.92539251A>G, 12:g.92539257C>T, 12:g.92539259G>A, 12:g.92539260C>T, 12:g.92539262G>A, 12:g.92539270C>G, 12:g.92539278T>G, 12:g.92539279C>T, 12:g.92539283G>A, 12:g.92539289G>T, 12:g.92539299A>G, 12:g.92539304_92539305delinsAC, 12:g.92539304G>A, 12:g.92539304G>C, 12:g.92539304G>T, 12:g.92539308G>A, 12:g.92539308G>T.

In some embodiment, the BTG1 inactivation results from a missense mutation selected in the group consisting in L26P, G66D, I115V, A127V, A130T, A17V, A18T, A18V, A49T, A83T, A84E, A84T, A84V, A8D, C149Y, C62F, C62Y, C69W, C69Y, D144H, D144Y, E101K, E117D, E126K, E14D, E34D, E46D, E46K, E46Q, E46V, E50K, E59D, F21L, F25C, F25I, F40C, G30E, G30R, G64E, G64R, G66R, G66V, G81R, H2N, H2Y, H51P, H54Y, H55R, H55Y, I115F, I121T, I12L, I22L, I22M, I22V, I70L, I70T, I70V, K162N, K24R, K29Q, K53R, K75N, L102I, L104H, L31F, L37M, L37V, L47Q, L47V, L94V, M11I, M166L, N165S, P3H, P3L, P3R, P3V, P61S, Q36H, Q38E, Q45P, R114I, R114K, R27H, R35G, R68H, R68L, S133N, S23A, S33N, S43N, T10I, T138S, T158A, T158M, T28S, T391, V1061, V111E, V111M, V143I, V19M, W56C, Y125C, Y5H, Y67H.

In some embodiment, the BTG1 inactivation results from a missense mutation selected in the group consisting in L26P, G66D and/or I115V.

In some embodiment, the present invention relates to a MCL-1 inhibitor for use in the treatment of a cancer in a patient in need thereof, wherein said patient carries a BTG1 gene inactivation.

As used herein, the term “treating” or “treatment” means reversing, alleviating, inhibiting the progress of, or preventing the disorder or condition to which such term applies, or reversing, alleviating, inhibiting the progress of, or preventing one or more symptoms of the disorder or condition to which such term applies.

By a “therapeutically effective amount” is meant a sufficient amount to be effective, at a reasonable benefit/risk ratio applicable to any medical treatment. It will be understood, however, that the total daily usage will be decided by the attending physician within the scope of sound medical judgment. The specific therapeutically effective dose level for any particular patient in need thereof will depend upon a variety of factors including the age, body weight, general health, sex and diet of the patient, the time of administration, route of administration, the duration of the treatment; drugs used in combination or coincidental with the and like factors well known in the medical arts. For example, it is well known within the skill of the art to start doses of the compound at levels lower than those required to achieve the desired therapeutic effect and to gradually increase the dosage until the desired effect is achieved.

The invention will be further illustrated by the following figures and examples. However, these examples and figures should not be interpreted in any way as limiting the scope of the present invention.

### FIGURES

FIG. 1: Graph representing BTG1 alteration frequencies depending upon cancer.

FIG. 2: Cellular models of BTG1 inactivation in ABC and GCB lymphoma cell lines. (A) Immunoblot of BTG1 in TMD8 cells, NT=Non-Targeting CRISPR, KO=BTG1 specific CRISPR, R=Re-expression of BTG1 using murine cDNA with 100% protein homology. (B) Immunoblot of BTG1 in SU-DHL-4 cells, NT=Non-Targeting CRISPR, KO=BTG1 specific CRISPR. (C) Representation of BTG1 sequence containing 3 specific boxes, representatives of the BTG family, and the location of the missense mutations.

FIG. 3: BTG1 inactivation is associated with increased sensitivity to MCL-1 inhibition. (A) Relative mitochondrial membrane potential loss using TMRE measurement (Relative apoptosis priming) of TMD8 BTG1 KO or TMD8 BTG1 KO with rescue as compared to BTG1 WT, after digitonin (5 μg/ml) and specific BH3 inhibitors treatment (20 μM each). (B) Proportion of apoptotic cells (Annexin V staining) after 72 h exposure to the MCL-1 inhibitor S63845 (0 or 50 nM) in TMD8 BTG1 KO, TMD8 BTG1 WT, TMD8 BTG1 Rescue or TMD8 BTG1 mutated rescues (striped) cell cultures. (C) Proportion of apoptotic cells (Annexin V staining) after 72 h exposure to the MCL-1 inhibitor S63845 (0 or 50 nM) in SUDHL-4 BTG1 KO or SUDHL-4 BTG1 WT cell cultures.

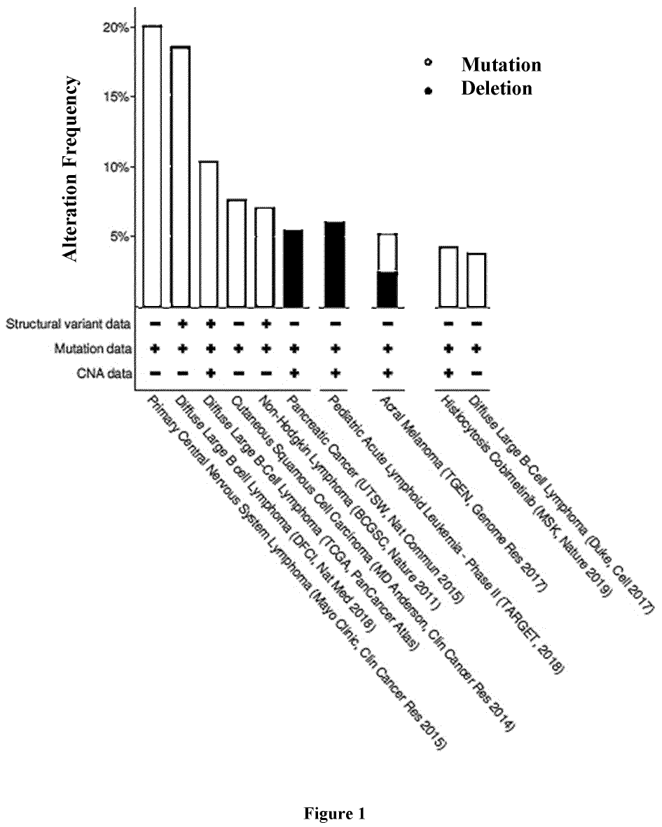

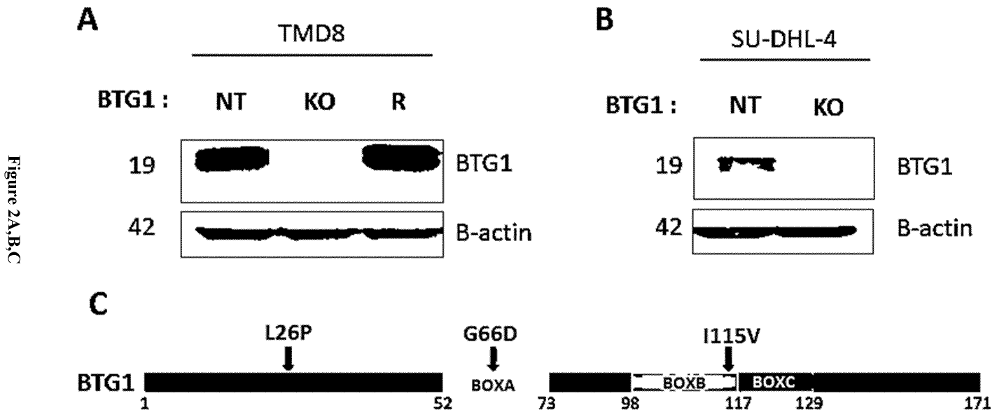

### EXAMPLE

### Material and Methods

### Cell Lines

TMD8 and SUDHL-4 were grown in Roswell Park Memorial Institute medium, GlutaMAX Supplement (61/870,044, Thermofisher), supplemented with 10% FBS, 1% penicillin G/streptomycin. Cells were grown in incubators at 37 C, in a 5% CO2 atmosphere.

### Generation of BTG1-KO Cell Lines

TMD8 and SUDHL-4 cells expressing cas9 were generated by transduction with lentiV-Cas9-blast virus (Addgene #125592, Massachusetts, USA), and selected with 2.5 μg/ml blasticidin for 7 days. Cells were then infected using pLKO.1-puro-GFP U6 sgRNA (Addgene #50920) containing BTG1 RNA guides or non-targeting RNA guides (target sequence for BTG1-KO-270: TTCTGTAGGACACTTCATAG (SEQ ID NO:2); BTG1-KO-355: AATGGATCCTCTGATTGGAC (SEQ ID NO:3); Non-Targeting-598: GCAGTACTACTGAGTTTTTC (SEQ ID NO:4); Non-Targeting-614: GCCTAGTCTCGGTAAGAGTG (SEQ ID NO:5)). Cells were selected with puromycin for 7 days, and GFP expression was evaluated with flow cytometry. BTG1 inactivation was validated by Western Blotting after 1 week. BTG1 rescue cell were derived from TMD8 BTG1 KO cells infected with murine BTG1 cDNA (100% protein homology with human) in a mcherry-HIV-based virus. GFP and mcherry positive cells were sorted on a FACS ARIA™ cell sorter (BD biosciences, San Jose, CA).

### BH3 Profiling

For BH3 profiling, 2×105 cells were washed with DTEB buffer (135 mM Trehalose, 20 μM EDTA, 20 μM EGTA, 5 mM Succinic Acid, 0.1% BSA, 10 mM HEPES, 50 mM KCl, pH 7.5) and permeabilized with DTEB+5 μg/mL digitonin. Loss of mitochondrial membrane potential (Wim) was assessed using TMRE (200 nM) after one hour of incubation at 30° C. with BH3 peptides as described previously (Del Gaizo Moore & Letai, 2013). TMRE is a cell permeant, positively charged, red-orange dye that readily accumulates in active mitochondria due to their relative negative charge. Depolarized or inactive mitochondria have decreased membrane potential and fail to sequester TMRE. The fluorescence was measured using a MACSQuant® Analyzers (Miltenyi, France).

### In Vitro Assay of MCL-1 Inhibitor

The high affinity MCL-1 specific inhibitor S63845 (Kotschy et al., 2016) was purchased from Selleck Chemical (München, Germany). For in vitro study, it was dissolved in DMSO at a concentration of lmmol/L. DMSO was also used as a vehicle control in the in vitro study. For cell viability assessment, 100 000 cells were seeded in 24 well plate and incubated for 72 h with either S63845 or DMSO. Culture medium was used for further dilutions (typically 5 and 50 nM). For cell viability assessment, cells were stained with Annexin V-APC and propidium iodide, analyzed on a FACS LSRII™ (BD biosciences, San Jose, CA).

### Results

We have established two cell lines models to study the impact of BTG1 inactivation. Using the CRISPR/Cas9 system, we have inactivated the BTG1 gene in the TMD8 cell line (FIG. 2A) (representative of the MCD/C5 subtype of DLBCL) and in the SUDHL4 cell line (FIG. 2B) (representative of the GC subtype of DLBCL). In the TMD8 cell line, we have rescued the expression of BTG1 with murine cDNA, using either a wild type sequence or a sequence with one of three missense mutations observed in patients (FIG. 2C).

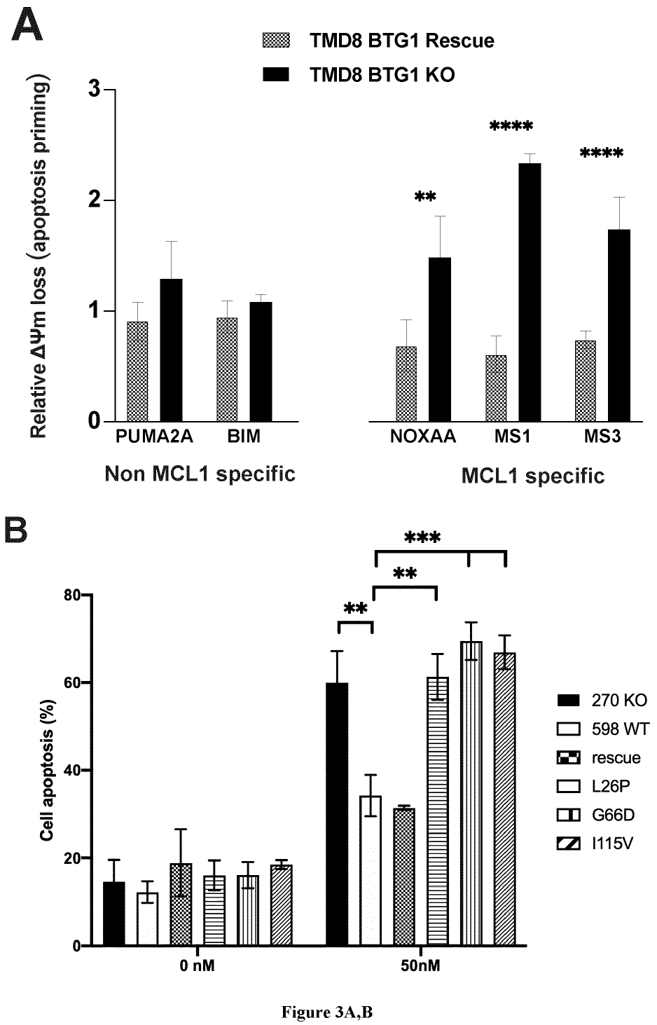

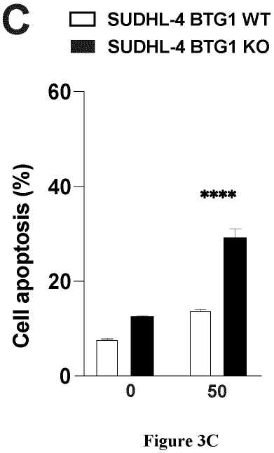

Using BH3 profiling, we have demonstrated in the TMD8 cell line that BTG1 inactivation creates an exquisite sensitivity to MCL-1 targeting peptides (such as NOXA, MS1 or MS3), and this phenomenon was fully reversed by the rescue of BTG1 expression. Of note, this differential effect was specific to MCL-1 targeting peptides, and not observed with peptides targeting other members of the BCL2 family (FIG. 3A). The link between BTG1 inactivation and increased sensitivity to MCL-1 inhibitors was further confirmed using the chemical inhibitor of MCL-1 S63845, which induces significantly more apoptosis in TMD8 BTG1 KO cells. Of note, the increase sensitivity to MCL-1 inhibitor was abolished by the expression of BTG1 WT, but not by the expression of mutated BTG1 engineered with a missense mutation described in patients (either L26P, G66D or I115V), (FIG. 3B). We confirmed the link between BTG1 inactivation and sensitivity to MCL-1 in the SUDHL4 cell line derived from a Germinal Centre DLBCL (FIG. 3C).

### CONCLUSION

Our results demonstrate that the inactivation of BTG1 creates a specific sensitivity to MCL-1 inhibitors, which suggests that clinical trials of MCL-1 inhibitors could be stratified according to the BTG1 mutational status of the cancer, where the therapeutic effects might be more pronounced.

### Tables

TABLE 1
Useful nucleotide and amino acid sequences for practicing the invention

| SEQ ID NO | Nucleotide or Amino Acid Sequence |
|---|---|
| 1 (BTG1 human gene cDNA) | atgcatccct tctacacceg ggccgccacc atgataggcg |
| | agategccgc cgccgtgtcc ttcatctcca agtttctccg |
| | caccaagggg ctcacgagcg agcgacagct |
| | gcagaccttc agccagagcc tgcaggagct |
| | gctggcagaa cattataaac atcactggtt cccagaaaag |
| | ccatgcaagg gatcgggtta ccgttgtatt cgcatcaacc |
| | ataaaatgga tcctctgatt ggacaggcag cacagoggat |
| | tggactgagc agtcaggagc tgttcaggct tctcccaagt |
| | gaactcacac tctgggttga cccctatgaa gtgtcctaca |
| | gaattggaga ggatggctcc atctgtgtgc tgtatgaagc |
| | ctcaccagca ggaggtagca ctcaaaacag |
| | caccaacgtg caaatggtag acagccgaat cagctgtaag |
| | gaggaacttc tettgggcag aacgagccct tccaaaaact |
| | acaatatgat gactgtatca ggttaa |
| 2 (target sequence for BTG1-KO-270) | ttctgtaggacacttcatag |
| 3 (target sequence for BTG1-KO-355) | aatggatcctctgattggac |
| 4 (Non-Targeting RNA Guide-598) | gcagtactactgagtttttc |
| 5 (Non-Targeting RNA Guide-614) | gcctagtctcggtaagagtg |

TABLE 2
most frequent mutations involved in BTG1 inactivation

| Cancer Type | Protein | | | |
|---|---|---|---|---|
| Detailed | Change | Mutation Type | HGVSg | Freq |
| Chronic | R35* | Nonsense_Mutation | 12:g.92539209G>A | 0.42 |
| Lymphocytic | | | | |
| Leukemia/ | | | | |
| Small | | | | |
| Lymphocytic | | | | |
| Lymphoma | | | | |
| Diffuse Large | M1? | Translation_Start_ | 12:g.92539309C>T | |
| B-Cell | | Site | | |
| Lymphoma, | | | | |
| NOS | | | | |
| Diffuse Large | X50_splice | Splice_Site | 12:g.92539163C>A | |
| B-Cell | | | | |
| Lymphoma, | | | | |
| NOS | | | | |
| Diffuse Large | W56* | Nonsense_Mutation | 12:g.92538204C>T | |
| B-Cell | | | | |
| Lymphoma, | | | | |
| NOS | | | | |
| Diffuse Large | W56* | Nonsense_Mutation | 12:g.92538204C>T | |
| B-Cell | | | | |
| Lymphoma, | | | | |
| NOS | | | | |
| Diffuse Large | Q45* | Nonsense_Mutation | 12:g.92539179G>A | |
| B-Cell | | | | |
| Lymphoma, | | | | |
| NOS | | | | |
| Diffuse Large | Q45* | Nonsense_Mutation | 12:g.92539179G>A | |
| B-Cell | | | | |
| Lymphoma, | | | | |
| NOS | | | | |
| Chronic | R35* | Nonsense_Mutation | 12:g.92539209G>A | |
| Lymphocytic | | | | |
| Leukemia/Small | | | | |
| Lymphocytic | | | | |
| Lymphoma | | | | |
| Diffuse Large | Q45Cfs*33 | Frame_Shift_Del | 12:g.92539174_92539180del | 0.40 |
| B-Cell | | | | |
| Lymphoma, | | | | |
| NOS | | | | |
| Diffuse Large | Q38* | Nonsense_Mutation | 12:g.92539200G>A | 0.15 |
| B-Cell | | | | |
| Lymphoma, | | | | |
| NOS | | | | |
| Diffuse Large | Q45Cfs*33 | Frame_Shift_Del | 12:g.92539174_92539180del | 0.23 |
| B-Cell | | | | |
| Lymphoma | | | | |
| Diffuse Large | Y67* | Nonsense_Mutation | 12:g.92538171G>C | 0.18 |
| B-Cell | | | | |
| Lymphoma | | | | |
| Diffuse Large | Q38* | Nonsense_Mutation | 12:g.92539200G>A | 0.14 |
| B-Cell | | | | |
| Lymphoma | | | | |
| Follicular | Q38* | Nonsense_Mutation | 12:g.92539200G>A | 0.27 |
| Lymphoma | | | | |
| Follicular | Q38* | Nonsense_Mutation | 12:g.92539200G>A | 0.38 |
| Lymphoma | | | | |
| Burkitt | K162* | Nonsense_Mutation | 12:g.92537888T>A | 0.42 |
| Lymphoma | | | | |
| Follicular | N136Kfs*9 | Frame_Shift_Ins | 12:g.92537964_92537965insTT | 0.31 |
| Lymphoma | | | | |
| Burkitt | W56* | Nonsense_Mutation | 12:g.92538204C>T | 0.44 |
| Lymphoma | | | | |
| Burkitt | W56* | Nonsense_Mutation | 12:g.92538204C>T | 0.31 |
| Lymphoma | | | | |
| Diffuse Large | W56* | Nonsense_Mutation | 12:g.92538205C>T | 0.36 |
| B-Cell | | | | |
| Lymphoma, | | | | |
| NOS | | | | |
| Diffuse Large | X50_splice | Splice_Region | 12:g.92538230_92538234del | 0.30 |
| B-Cell | | | | |
| Lymphoma, | | | | |
| NOS | | | | |
| High-Grade | X50_splice | Splice_Region | 12:g.92539158G>A | 0.45 |
| B-Cell | | | | |
| Lymphoma, | | | | |
| with MYC | | | | |
| and BCL2 | | | | |
| and/or BCL6 | | | | |
| Rearrangements | | | | |
| Diffuse Large | X50_splice | Splice_Region | 12:g.92539159C>A | 0.30 |
| B-Cell | | | | |
| Lymphoma, | | | | |
| NOS | | | | |
| Follicular | X50_splice | Splice_Region | 12:g.92539159C>T | 0.22 |
| Lymphoma | | | | |
| Follicular | K29 | Nonsense_Mutation | 12:g.92539227T>A | 0.17 |
| Lymphoma | | | | |
| Burkitt | M1? | Translation_Start_Site | 12:g.92539309C>T | 0.34 |
| Lymphoma | | | | |
| Follicular | M1? | Translation_Start_Site | 12:g.92539309C>T | 0.21 |
| Lymphoma | | | | |
| Follicular | M1? | Translation_Start_Site | 12:g.92539309C>T | 0.14 |
| Lymphoma | | | | |
| Germinal | W56* | Nonsense_Mutation | 12:g.92538204C>T | 0.20 |
| Center B-Cell | | | | |
| Type | | | | |
| Diffuse Large | X50_splice | Splice_Site | 12:g.92539163C>A | 0.37 |
| B-Cell | | | | |
| Lymphoma, | | | | |
| NOS | | | | |
| Diffuse Large | *172Sext*9 | Nonstop_Mutation | 12:g.92537857T>G | 0.22 |
| B-Cell | | | | |
| Lymphoma, | | | | |
| NOS | | | | |
| Chronic | E34D | Missense_Mutation | 12:g.92539210C>G | 0.12 |
| Lymphocytic | | | | |
| Leukemia/Small | | | | |
| Lymphocytic | | | | |
| Lymphoma | | | | |
| Diffuse Large | N165S | Missense_Mutation | 12:g.92537878T>C | |
| B-Cell | | | | |
| Lymphoma, | | | | |
| NOS | | | | |
| Diffuse Large | P3R | Missense_Mutation | 12:g.92539304G>C | |
| B-Cell | | | | |
| Lymphoma, | | | | |
| NOS | | | | |
| Diffuse Large | Y5H | Missense_Mutation | 12:g.92539299A>G | |
| B-Cell | | | | |
| Lymphoma, | | | | |
| NOS | | | | |
| Diffuse Large | L31F | Missense_Mutation | 12:g.92539221G>A | |
| B-Cell | | | | |
| Lymphoma, | | | | |
| NOS | | | | |
| Diffuse Large | L31F | Missense_Mutation | 12:g.92539221G>A | |
| B-Cell | | | | |
| Lymphoma, | | | | |
| NOS | | | | |
| Diffuse Large | M11I | Missense_Mutation | 12:g.92539279C>T | |
| B-Cell | | | | |
| Lymphoma, | | | | |
| NOS | | | | |
| Diffuse Large | E46Q | Missense_Mutation | 12:g.92539176C>G | |
| B-Cell | | | | |
| Lymphoma, | | | | |
| NOS | | | | |
| Diffuse Large | K29Q | Missense_Mutation | 12:g.92539227T>G | |
| B-Cell | | | | |
| Lymphoma, | | | | |
| NOS | | | | |
| Diffuse Large | F25C | Missense_Mutation | 12:g.92539238A>C | |
| B-Cell | | | | |
| Lymphoma, | | | | |
| NOS | | | | |
| Diffuse Large | S23A | Missense_Mutation | 12:g.92539245A>C | |
| B-Cell | | | | |
| Lymphoma, | | | | |
| NOS | | | | |
| Diffuse Large | E59D | Missense_Mutation | 12:g.92538195T>A | |
| B-Cell | | | | |
| Lymphoma, | | | | |
| NOS | | | | |
| Diffuse Large | I115V | Missense_Mutation | 12:g.92538029T>C | |
| B-Cell | | | | |
| Lymphoma, | | | | |
| NOS | | | | |
| Diffuse Large | F40C | Missense_Mutation | 12:g.92539193A>C | |
| B-Cell | | | | |
| Lymphoma, | | | | |
| NOS | | | | |
| Diffuse Large | E117D | Missense_Mutation | 12:g.92538021C>A | |
| B-Cell | | | | |
| Lymphoma, | | | | |
| NOS | | | | |
| Diffuse Large | A18V | Missense_Mutation | 12:g.92539259G>A | |
| B-Cell | | | | |
| Lymphoma, | | | | |
| NOS | | | | |
| Diffuse Large | G30E | Missense_Mutation | 12:g.92539223C>T | |
| B-Cell | | | | |
| Lymphoma, | | | | |
| NOS | | | | |
| Diffuse Large | G30E | Missense_Mutation | 12:g.92539223C>T | |
| B-Cell | | | | |
| Lymphoma, | | | | |
| NOS | | | | |
| Diffuse Large | S43N | Missense_Mutation | 12:g.92539184C>T | |
| B-Cell | | | | |
| Lymphoma, | | | | |
| NOS | | | | |
| Diffuse Large | S43N | Missense_Mutation | 12:g.92539184C>T | |
| B-Cell | | | | |
| Lymphoma, | | | | |
| NOS | | | | |
| Diffuse Large | T158A | Missense_Mutation | 12:g.92537900T>C | |
| B-Cell | | | | |
| Lymphoma, | | | | |
| NOS | | | | |
| Diffuse Large | T138S | Missense_Mutation | 12:g.92537960T>A | |
| B-Cell | | | | |
| Lymphoma, | | | | |
| NOS | | | | |
| Diffuse Large | T39I | Missense_Mutation | 12:g.92539196G>A | |
| B-Cell | | | | |
| Lymphoma, | | | | |
| NOS | | | | |
| Diffuse Large | T39I | Missense_Mutation | 12:g.92539196G>A | |
| B-Cell | | | | |
| Lymphoma, | | | | |
| NOS | | | | |
| Diffuse Large | S33N | Missense_Mutation | 12:g.92539214C>T | |
| B-Cell | | | | |
| Lymphoma, | | | | |
| NOS | | | | |
| Diffuse Large | L31F | Missense_Mutation | 12:g.92539221G>A | |
| B-Cell | | | | |
| Lymphoma, | | | | |
| NOS | | | | |
| Diffuse Large | M11I | Missense_Mutation | 12:g.92539279C>T | |
| B-Cell | | | | |
| Lymphoma, | | | | |
| NOS | | | | |
| Diffuse Large | M11I | Missense_Mutation | 12:g.92539279C>T | |
| B-Cell | | | | |
| Lymphoma, | | | | |
| NOS | | | | |
| Diffuse Large | P3L | Missense_Mutation | 12:g.92539304G>A | |
| B-Cell | | | | |
| Lymphoma, | | | | |
| NOS | | | | |
| Diffuse Large | P3L | Missense_Mutation | 12:g.92539304G>A | |
| B-Cell | | | | |
| Lymphoma, | | | | |
| NOS | | | | |
| Diffuse Large | P3L | Missense_Mutation | 12:g.92539304G>A | |
| B-Cell | | | | |
| Lymphoma, | | | | |
| NOS | | | | |
| Diffuse Large | P3L | Missense_Mutation | 12:g.92539304G>A | |
| B-Cell | | | | |
| Lymphoma, | | | | |
| NOS | | | | |
| Diffuse Large | G66D | Missense_Mutation | 12:g.92538175C>T | |
| B-Cell | | | | |
| Lymphoma, | | | | |
| NOS | | | | |
| Diffuse Large | C69Y | Missense_Mutation | 12:g.92538166C>T | |
| B-Cell | | | | |
| Lymphoma, | | | | |
| NOS | | | | |
| Diffuse Large | G30R | Missense_Mutation | 12:g.92539224C>T | |
| B-Cell | | | | |
| Lymphoma, | | | | |
| NOS | | | | |
| Diffuse Large | H2Y | Missense_Mutation | 12:g.92539308G>A | |
| B-Cell | | | | |
| Lymphoma, | | | | |
| NOS | | | | |
| Diffuse Large | H2Y | Missense_Mutation | 12:g.92539308G>A | |
| B-Cell | | | | |
| Lymphoma, | | | | |
| NOS | | | | |
| Diffuse Large | H2Y | Missense_Mutation | 12:g.92539308G>A | |
| B-Cell | | | | |
| Lymphoma, | | | | |
| NOS | | | | |
| Diffuse Large | M166L | Missense_Mutation | 12:g.92537876T>A | |
| B-Cell | | | | |
| Lymphoma, | | | | |
| NOS | | | | |
| Diffuse Large | D144Y | Missense_Mutation | 12:g.92537942C>A | |
| B-Cell | | | | |
| Lymphoma, | | | | |
| NOS | | | | |
| Diffuse Large | S133N | Missense_Mutation | 12:g.92537974C>T | |
| B-Cell | | | | |
| Lymphoma, | | | | |
| NOS | | | | |
| Diffuse Large | I121T | Missense_Mutation | 12:g.92538010A>G | |
| B-Cell | | | | |
| Lymphoma, | | | | |
| NOS | | | | |
| Diffuse Large | I115F | Missense_Mutation | 12:g.92538029T>A | |
| B-Cell | | | | |
| Lymphoma, | | | | |
| NOS | | | | |
| Diffuse Large | V106I | Missense_Mutation | 12:g.92538056C>T | |
| B-Cell | | | | |
| Lymphoma, | | | | |
| NOS | | | | |
| Diffuse Large | L104H | Missense_Mutation | 12:g.92538061A>T | |
| B-Cell | | | | |
| Lymphoma, | | | | |
| NOS | | | | |
| Diffuse Large | A84V | Missense_Mutation | 12:g.92538121G>A | |
| B-Cell | | | | |
| Lymphoma, | | | | |
| NOS | | | | |
| Diffuse Large | 170V | Missense_Mutation | 12:g.92538164T>C | |
| B-Cell | | | | |
| Lymphoma, | | | | |
| NOS | | | | |
| Diffuse Large | Y67H | Missense_Mutation | 12:g.92538173A>G | |
| B-Cell | | | | |
| Lymphoma, | | | | |
| NOS | | | | |
| Diffuse Large | G64R | Missense_Mutation | 12:g.92538182C>T | |
| B-Cell | | | | |
| Lymphoma, | | | | |
| NOS | | | | |
| Diffuse Large | P61S | Missense_Mutation | 12:g.92538191G>A | |
| B-Cell | | | | |
| Lymphoma, | | | | |
| NOS | | | | |
| Diffuse Large | H54Y | Missense_Mutation | 12:g.92538212G>A | |
| B-Cell | | | | |
| Lymphoma, | | | | |
| NOS | | | | |
| Diffuse Large | H51P | Missense_Mutation | 12:g.92538220T>G | |
| B-Cell | | | | |
| Lymphoma, | | | | |
| NOS | | | | |
| Diffuse Large | Q45P | Missense_Mutation | 12:g.92539178T>G | |
| B-Cell | | | | |
| Lymphoma, | | | | |
| NOS | | | | |
| Diffuse Large | Q45P | Missense_Mutation | 12:g.92539178T>G | |
| B-Cell | | | | |
| Lymphoma, | | | | |
| NOS | | | | |
| Diffuse Large | L26P | Missense_Mutation | 12:g.92539235A>G | |
| B-Cell | | | | |
| Lymphoma, | | | | |
| NOS | | | | |
| Diffuse Large | V19M | Missense_Mutation | 12:g.92539257C>T | |
| B-Cell | | | | |
| Lymphoma, | | | | |
| NOS | | | | |
| Diffuse Large | A18T | Missense_Mutation | 12:g.92539260C>T | |
| B-Cell | | | | |
| Lymphoma, | | | | |
| NOS | | | | |
| Diffuse Large | E14D | Missense_Mutation | 12:g.92539270C>G | |
| B-Cell | | | | |
| Lymphoma, | | | | |
| NOS | | | | |
| Diffuse Large | E14D | Missense_Mutation | 12:g.92539270C>G | |
| B-Cell | | | | |
| Lymphoma, | | | | |
| NOS | | | | |
| Diffuse Large | S43N | Missense_Mutation | 12:g.92539184C>T | |
| B-Cell | | | | |
| Lymphoma, | | | | |
| NOS | | | | |
| Diffuse Large | T10I | Missense_Mutation | 12:g.92539283G>A | |
| B-Cell | | | | |
| Lymphoma, | | | | |
| NOS | | | | |
| Plasma Cell | T28S | Missense_Mutation | 12:g.92539229G>C | |
| Myeloma | | | | |
| Plasma Cell | P3V | Missense_Mutation | 12:g.92539304_92539305delins | |
| Myeloma | | | AC | |
| Plasma Cell | K53R | Missense_Mutation | 12:g.92538214T>C | |
| Myeloma | | | | |
| Germinal | L94V | Missense_Mutation | 12:g.92538092G>C | |
| Center B-Cell | | | | |
| Type | | | | |
| Diffuse Large | E46D | Missense_Mutation | 12:g.92539174C>G | 0.52 |
| B-Cell | | | | |
| Lymphoma, | | | | |
| NOS | | | | |
| Diffuse Large | C62Y | Missense_Mutation | 12:g.92538187C>T | 0.30 |
| B-Cell | | | | |
| Lymphoma, | | | | |
| NOS | | | | |
| Diffuse Large | P3R | Missense_Mutation | 12:g.92539304G>C | 0.45 |
| B-Cell | | | | |
| Lymphoma, | | | | |
| NOS | | | | |
| Diffuse Large | T158M | Missense_Mutation | 12:g.92537899G>A | 0.12 |
| B-Cell | | | | |
| Lymphoma, | | | | |
| NOS | | | | |
| Diffuse Large | A127V | Missense_Mutation | 12:g.92537992G>A | 0.20 |
| B-Cell | | | | |
| Lymphoma, | | | | |
| NOS | | | | |
| Diffuse Large | E46D | Missense_Mutation | 12:g.92539174C>G | 0.52 |
| B-Cell | | | | |
| Lymphoma | | | | |
| Diffuse Large | C62Y | Missense_Mutation | 12:g.92538187C>T | 0.30 |
| B-Cell | | | | |
| Lymphoma | | | | |
| Diffuse Large | P3R | Missense_Mutation | 12:g.92539304G>C | 0.47 |
| B-Cell | | | | |
| Lymphoma | | | | |
| Diffuse Large | G30E | Missense_Mutation | 12:g.92539223C>T | 0.32 |
| B-Cell | | | | |
| Lymphoma | | | | |
| Diffuse Large | T158M | Missense_Mutation | 12:g.92537899G>A | 0.12 |
| B-Cell | | | | |
| Lymphoma | | | | |
| Diffuse Large | A127V | Missense_Mutation | 12:g.92537992G>A | 0.21 |
| B-Cell | | | | |
| Lymphoma | | | | |
| Diffuse Large | G30E | Missense_Mutation | 12:g.92539223C>T | 0.26 |
| B-Cell | | | | |
| Lymphoma, | | | | |
| NOS | | | | |
| Burkitt | S43N | Missense_Mutation | 12:g.92539184C>T | 0.28 |
| Lymphoma | | | | |
| Follicular | H55R | Missense_Mutation | 12:g.92538208T>C | 0.16 |
| Lymphoma | | | | |
| Burkitt | T158A | Missense_Mutation | 12:g.92537900T>C | 0.43 |
| Lymphoma | | | | |
| Follicular | C149Y | Missense_Mutation | 12:g.92537926C>T | 0.22 |
| Lymphoma | | | | |
| Burkitt | D144H | Missense_Mutation | 12:g.92537942C>G | 0.21 |
| Lymphoma | | | | |
| Diffuse Large | V143I | Missense_Mutation | 12:g.92537945C>T | 0.33 |
| B-Cell | | | | |
| Lymphoma, | | | | |
| NOS | | | | |
| High-Grade | V143I | Missense_Mutation | 12:g.92537945C>T | 0.29 |
| B-Cell | | | | |
| Lymphoma, | | | | |
| NOS | | | | |
| Diffuse Large | T138S | Missense_Mutation | 12:g.92537960T>A | 0.20 |
| B-Cell | | | | |
| Lymphoma, | | | | |
| NOS | | | | |
| Diffuse Large | R114I | Missense_Mutation | 12:g.92538031C>A | 0.33 |
| B-Cell | | | | |
| Lymphoma, | | | | |
| NOS | | | | |
| Diffuse Large | R114K | Missense_Mutation | 12:g.92538031C>T | 0.15 |
| B-Cell | | | | |
| Lymphoma, | | | | |
| NOS | | | | |
| Diffuse Large | L102I | Missense_Mutation | 12:g.92538068G>T | 0.11 |
| B-Cell | | | | |
| Lymphoma, | | | | |
| NOS | | | | |
| Diffuse Large | E101K | Missense_Mutation | 12:g.92538071C>T | 0.14 |
| B-Cell | | | | |
| Lymphoma, | | | | |
| NOS | | | | |
| High-Grade | A84E | Missense_Mutation | 12:g.92538121G>T | 0.42 |
| B-Cell | | | | |
| Lymphoma, | | | | |
| with MYC | | | | |
| and BCL2 | | | | |
| and/or BCL6 | | | | |
| Rearrangements | | | | |
| Diffuse Large | A84T | Missense_Mutation | 12:g.92538122C>T | 0.32 |
| B-Cell | | | | |
| Lymphoma, | | | | |
| NOS | | | | |
| Diffuse Large | A83T | Missense_Mutation | 12:g.92538125C>T | 0.34 |
| B-Cell | | | | |
| Lymphoma, | | | | |
| NOS | | | | |
| Burkitt | G81R | Missense_Mutation | 12:g.92538131C>T | 0.20 |
| Lymphoma | | | | |
| Diffuse Large | K75N | Missense_Mutation | 12:g.92538147T>G | 0.20 |
| B-Cell | | | | |
| Lymphoma, | | | | |
| NOS | | | | |
| High-Grade | I70T | Missense_Mutation | 12:g.92538163A>G | 0.40 |
| B-Cell | | | | |
| Lymphoma, | | | | |
| with MYC | | | | |
| and BCL2 | | | | |
| and/or BCL6 | | | | |
| Rearrangements | | | | |
| High-Grade | C69W | Missense_Mutation | 12:g.92538165A>C | 0.41 |
| B-Cell | | | | |
| Lymphoma, | | | | |
| with MYC | | | | |
| and BCL2 | | | | |
| and/or BCL6 | | | | |
| Rearrangements | | | | |
| Diffuse Large | R68L | Missense_Mutation | 12:g.92538169C>A | 0.31 |
| B-Cell | | | | |
| Lymphoma, | | | | |
| NOS | | | | |
| Diffuse Large | G66R | Missense_Mutation | 12:g.92538176C>G | 0.13 |
| B-Cell | | | | |
| Lymphoma, | | | | |
| NOS | | | | |
| Diffuse Large | G64E | Missense_Mutation | 12:g.92538181C>T | 0.33 |
| B-Cell | | | | |
| Lymphoma, | | | | |
| NOS | | | | |
| Diffuse Large | C62F | Missense_Mutation | 12:g.92538187C>A | 0.13 |
| B-Cell | | | | |
| Lymphoma, | | | | |
| NOS | | | | |
| Diffuse Large | W56C | Missense_Mutation | 12:g.92538204C>G | 0.38 |
| B-Cell | | | | |
| Lymphoma, | | | | |
| NOS | | | | |
| Diffuse Large | W56C | Missense_Mutation | 12:g.92538204C>G | 0.37 |
| B-Cell | | | | |
| Lymphoma, | | | | |
| NOS | | | | |
| Diffuse Large | H55Y | Missense_Mutation | 12:g.92538209G>A | 0.31 |
| B-Cell | | | | |
| Lymphoma, | | | | |
| NOS | | | | |
| Diffuse Large | H55Y | Missense_Mutation | 12:g.92538209G>A | 0.36 |
| B-Cell | | | | |
| Lymphoma, | | | | |
| NOS | | | | |
| High-Grade | H55Y | Missense_Mutation | 12:g.92538209G>A | 0.27 |
| B-Cell | | | | |
| Lymphoma, | | | | |
| NOS | | | | |
| Follicular | E50K | Missense_Mutation | 12:g.92539164C>T | 0.26 |
| Lymphoma | | | | |
| Diffuse Large | A49T | Missense_Mutation | 12:g.92539167C>T | 0.61 |
| B-Cell | | | | |
| Lymphoma, | | | | |
| NOS | | | | |
| Follicular | A49T | Missense_Mutation | 12:g.92539167C>T | 0.15 |
| Lymphoma | | | | |
| Diffuse Large | T39I | Missense_Mutation | 12:g.92539196G>A | 0.28 |
| B-Cell | | | | |
| Lymphoma, | | | | |
| NOS | | | | |
| Diffuse Large | Q38E | Missense_Mutation | 12:g.92539200G>C | 0.18 |
| B-Cell | | | | |
| Lymphoma, | | | | |
| NOS | | | | |
| Diffuse Large | Q36H | Missense_Mutation | 12:g.92539204C>G | 0.11 |
| B-Cell | | | | |
| Lymphoma, | | | | |
| NOS | | | | |
| Diffuse Large | Q36H | Missense_Mutation | 12:g.92539204C>G | 0.32 |
| B-Cell | | | | |
| Lymphoma, | | | | |
| NOS | | | | |
| High-Grade | Q36H | Missense_Mutation | 12:g.92539204C>G | 0.45 |
| B-Cell | | | | |
| Lymphoma, | | | | |
| NOS | | | | |
| Diffuse Large | R35G | Missense_Mutation | 12:g.92539209G>C | 0.12 |
| B-Cell | | | | |
| Lymphoma, | | | | |
| NOS | | | | |
| High-Grade | S33N | Missense_Mutation | 12:g.92539214C>T | 0.42 |
| B-Cell | | | | |
| Lymphoma, | | | | |
| NOS | | | | |
| Burkitt | L31F | Missense_Mutation | 12:g.92539221G>A | 0.22 |
| Lymphoma | | | | |
| Diffuse Large | L31F | Missense_Mutation | 12:g.92539221G>A | 0.45 |
| B-Cell | | | | |
| Lymphoma, | | | | |
| NOS | | | | |
| Burkitt | R27H | Missense_Mutation | 12:g.92539232C>T | 0.33 |
| Lymphoma | | | | |
| Diffuse Large | F25I | Missense_Mutation | 12:g.92539239A>T | 0.14 |
| B-Cell | | | | |
| Lymphoma, | | | | |
| NOS | | | | |
| Follicular | K24R | Missense_Mutation | 12:g.92539241T>C | 0.25 |
| Lymphoma | | | | |
| Diffuse Large | I22M | Missense_Mutation | 12:g.92539246G>C | 0.32 |
| B-Cell | | | | |
| Lymphoma, | | | | |
| NOS | | | | |
| Diffuse Large | I22L | Missense_Mutation | 12:g.92539248T>G | 0.25 |
| B-Cell | | | | |
| Lymphoma, | | | | |
| NOS | | | | |
| Diffuse Large | I22V | Missense_Mutation | 12:g.92539248T>C | 0.56 |
| B-Cell | | | | |
| Lymphoma, | | | | |
| NOS | | | | |
| Diffuse Large | F21L | Missense_Mutation | 12:g.92539251A>G | 0.13 |
| B-Cell | | | | |
| Lymphoma, | | | | |
| NOS | | | | |
| Follicular | F21L | Missense_Mutation | 12:g.92539251A>G | 0.19 |
| Lymphoma | | | | |
| Diffuse Large | A17V | Missense_Mutation | 12:g.92539262G>A | 0.37 |
| B-Cell | | | | |
| Lymphoma, | | | | |
| NOS | | | | |
| Follicular | I12L | Missense_Mutation | 12:g.92539278T>G | 0.29 |
| Lymphoma | | | | |
| Diffuse Large | M11I | Missense_Mutation | 12:g.92539279C>T | 0.31 |
| B-Cell | | | | |
| Lymphoma, | | | | |
| NOS | | | | |
| Diffuse Large | P3L | Missense_Mutation | 12:g.92539304G>A | 0.15 |
| B-Cell | | | | |
| Lymphoma, | | | | |
| NOS | | | | |
| Follicular | P3L | Missense_Mutation | 12:g.92539304G>A | 0.17 |
| Lymphoma | | | | |
| Diffuse Large | H2N | Missense_Mutation | 12:g.92539308G>T | 0.16 |
| B-Cell | | | | |
| Lymphoma, | | | | |
| NOS | | | | |
| Chronic | L37M | Missense_Mutation | 12:g.92539203G>T | |
| Lymphocytic | | | | |
| Leukemia/Small | | | | |
| Lymphocytic | | | | |
| Lymphoma | | | | |
| Chronic | P3H | Missense_Mutation | 12:g.92539304G>T | |
| Lymphocytic | | | | |
| Leukemia/Small | | | | |
| Lymphocytic | | | | |
| Lymphoma | | | | |
| Germinal | L94V | Missense_Mutation | 12:g.92538092G>C | |
| Center B-Cell | | | | |
| Type | | | | |
| Diffuse Large | N165S | Missense_Mutation | 12:g.92537878T>C | 0.31 |
| B-Cell | | | | |
| Lymphoma, | | | | |
| NOS | | | | |
| Diffuse Large | E46D | Missense_Mutation | 12:g.92539174C>G | 0.08 |
| B-Cell | | | | |
| Lymphoma, | | | | |
| NOS | | | | |
| Activated B- | P3R | Missense_Mutation | 12:g.92539304G>C | 0.26 |
| cell Type | | | | |
| Activated B- | S43N | Missense_Mutation | 12:g.92539184C>T | 0.19 |
| cell Type | | | | |
| Germinal | Y5H | Missense_Mutation | 12:g.92539299A>G | 0.20 |
| Center B-Cell | | | | |
| Type | | | | |
| Diffuse Large | I70T | Missense_Mutation | 12:g.92538163A>G | 0.06 |
| B-Cell | | | | |
| Lymphoma, | | | | |
| NOS | | | | |
| Diffuse Large | L31F | Missense_Mutation | 12:g.92539221G>A | 0.18 |
| B-Cell | | | | |
| Lymphoma, | | | | |
| NOS | | | | |
| Activated B- | L31F | Missense_Mutation | 12:g.92539221G>A | 0.32 |
| cell Type | | | | |
| Germinal | I22M | Missense_Mutation | 12:g.92539246G>C | 0.26 |
| Center B-Cell | | | | |
| Type | | | | |
| Activated B- | M11I | Missense_Mutation | 12:g.92539279C>T | 0.41 |
| cell Type | | | | |
| Diffuse Large | V111M | Missense_Mutation | 12:g.92538041C>T | 0.20 |
| B-Cell | | | | |
| Lymphoma, | | | | |
| NOS | | | | |
| Activated B- | C69Y | Missense_Mutation | 12:g.92538166C>T | 0.23 |
| cell Type | | | | |
| Diffuse Large | G30R | Missense_Mutation | 12:g.92539224C>T | 0.09 |
| B-Cell | | | | |
| Lymphoma, | | | | |
| NOS | | | | |
| Germinal | H2Y | Missense_Mutation | 12:g.92539308G>A | 0.29 |
| Center B-Cell | | | | |
| Type | | | | |
| Diffuse Large | V111E | Missense_Mutation | 12:g.92538040A>T | 0.07 |
| B-Cell | | | | |
| Lymphoma, | | | | |
| NOS | | | | |
| Activated B- | E46Q | Missense_Mutation | 12:g.92539176C>G | 0.32 |
| cell Type | | | | |
| Diffuse Large | K29Q | Missense_Mutation | 12:g.92539227T>G | 0.55 |
| B-Cell | | | | |
| Lymphoma, | | | | |
| NOS | | | | |
| Germinal | F25C | Missense_Mutation | 12:g.92539238A>C | 0.24 |
| Center B-Cell | | | | |
| Type | | | | |
| Germinal | S23A | Missense_Mutation | 12:g.92539245A>C | 0.20 |
| Center B-Cell | | | | |
| Type | | | | |
| Diffuse Large | E59D | Missense_Mutation | 12:g.92538195T>A | 0.50 |
| B-Cell | | | | |
| Lymphoma, | | | | |
| NOS | | | | |
| Diffuse Large | I115V | Missense_Mutation | 12:g.92538029T>C | 0.25 |
| B-Cell | | | | |
| Lymphoma, | | | | |
| NOS | | | | |
| Diffuse Large | F40C | Missense_Mutation | 12:g.92539193A>C | 0.37 |
| B-Cell | | | | |
| Lymphoma, | | | | |
| NOS | | | | |
| Germinal | E117D | Missense_Mutation | 12:g.92538021C>A | 0.22 |
| Center B-Cell | | | | |
| Type | | | | |
| Diffuse Large | K162N | Missense_Mutation | 12:g.92537886T>G | 0.11 |
| B-Cell | | | | |
| Lymphoma, | | | | |
| NOS | | | | |
| Activated B- | I70L | Missense_Mutation | 12:g.92538164T>G | 0.08 |
| cell Type | | | | |
| Activated B- | Y125C | Missense_Mutation | 12:g.92537998T>C | 0.06 |
| cell Type | | | | |
| Activated B- | E46K | Missense_Mutation | 12:g.92539176C>T | 0.36 |
| cell Type | | | | |
| Activated B- | R68H | Missense_Mutation | 12:g.92538169C>T | 0.04 |
| cell Type | | | | |
| Diffuse Large | E126K | Missense_Mutation | 12:g.92537996C>T | 0.17 |
| B-Cell | | | | |
| Lymphoma, | | | | |
| NOS | | | | |
| Germinal | E46V | Missense_Mutation | 12:g.92539175T>A | 0.29 |
| Center B-Cell | | | | |
| Type | | | | |
| Germinal | E46D | Missense_Mutation | 12:g.92539174C>A | 0.30 |
| Center B-Cell | | | | |
| Type | | | | |
| Diffuse Large | G66V | Missense_Mutation | 12:g.92538175C>A | 0.30 |
| B-Cell | | | | |
| Lymphoma, | | | | |
| NOS | | | | |
| Diffuse Large | A130T | Missense_Mutation | 12:g.92537984C>T | 0.04 |
| B-Cell | | | | |
| Lymphoma, | | | | |
| NOS | | | | |
| Activated B- | L37V | Missense_Mutation | 12:g.92539203G>C | 0.10 |
| cell Type | | | | |
| Activated B- | L47V | Missense_Mutation | 12:g. 92539173G>C | 0.24 |
| cell Type | | | | |
| Activated B- | A8D | Missense_Mutation | 12:g.92539289G>T | 0.16 |
| cell Type | | | | |
| Activated B- | L47Q | Missense_Mutation | 12:g.92539172A>T | 0.23 |
| cell Type | | | | |

?Translation Start Site
*Stop Codon
Source: https://www.cbioportal.org/

### REFERENCES

Almasmoum, H. A., Airhihen, B., Seedhouse, C., & Winkler, G. S. (2020). Frequent loss of BTG1 activity and impaired interactions with the Caf1 subunit of the Ccr4-Not deadenylase in non-Hodgkin lymphoma. Leukemia and Lymphoma, 0, 1-10.
Amin, A. D., Peters, T. L., Li, L., Rajan, S. S., Choudhari, R., Puvvada, S. D., & Schatz, J. H. (2017). Diffuse large B-cell lymphoma: can genomics improve treatment options for a curable cancer? Molecular Case Studies, 3, a001719.
Berthet, C., Guehenneux, F., Revol, V., Samarut, C., Lukaszewicz, A., Dehay, C., . . . Rouault, J.-P. (2002). Interaction of PRMT1 with BTG/TOB proteins in cell signalling: molecular analysis and functional aspects. Genes to Cells: Devoted to Molecular & Cellular Mechanisms, 7, 29-39.
Chapuy, B., Stewart, C., Dunford, A. J., Kim, J., Kamburov, A., Redd, R. A., . . . Shipp, M. A. (2018). Molecular subtypes of diffuse large B cell lymphoma are associated with distinct pathogenic mechanisms and outcomes. Nature Medicine, 24, 679-690.
Del Gaizo Moore, V., & Letai, A. (2013). BH3 profiling—Measuring integrated function of the mitochondrial apoptotic pathway to predict cell fate decisions. Cancer Letters, 332, 202-205.
Duke, J. L., Liu, M., Yaari, G., Khalil, A. M., Tomayko, M., Shlomchik, M. J., . . . Kleinstein, S. H. (2013). Multiple Transcription Factor Binding Sites Predict AID Targeting in Non-Ig Genes. The Journal of Immunology. doi:10.4049/jimmunol.1202547
Hunter, Z. R., Xu, L., Yang, G., Zhou, Y., Liu, X., Cao, Y., . . . Treon, S. P. (2014). The genomic landscape of Waldenstrom macroglobulinemia is characterized by highly recurring MYD88 and WHIM-like CXCR4 mutations, and small somatic deletions associated with B-cell lymphomagenesis. Blood, 123, 1637-1646.
Kotschy, A., Szlavik, Z., Murray, J., Davidson, J., Maragno, A. L., Le Toumelin-Braizat, G., . . . Geneste, O. (2016). The MCL-1 inhibitor S63845 is tolerable and effective in diverse cancer models. Nature, 538, 477-482.
Lin, W. J., Gary, J. D., Yang, M. C., Clarke, S., & Herschman, H. R. (1996). The mammalian immediate-early TIS21 protein and the leukemia-associated BTG1 protein interact with a protein-arginine N-methyltransferase. Journal of Biological Chemistry, 271, 15034-15044.
Reddy, A., Zhang, J., Davis, N. S., Moffitt, A. B., Love, C. L., Waldrop, A., . . . Dave, S. S. (2017). Genetic and Functional Drivers of Diffuse Large B Cell Lymphoma. Cell, 171, 481-494.e15.
Rouault, J.-P., Prevot, D., Berthet, C., Birot, A.-M., Billaud, M., Magaud, J.-P., & Corbo, L. (1998). Interaction of BTG1 and p53-regulated BTG2 Gene Products with mCafl, the Murine Homolog of a Component of the Yeast CCR4 Transcriptional Regulatory Complex. Journal of Biological Chemistry, 273, 22563-22569.
Rouault, J. P., Rimokh, R., Tessa, C., Paranhos, G., Ffrench, M., Duret, L., . . . Magaud, J. P. (1992). BTG1, a member of a new family of antiproliferative genes. The EMBO Journal, 11, 1663-70.
Schmitz, R., Wright, G. W., Huang, D. W., Johnson, C. A., Phelan, J. D., Wang, J. Q., . . . Staudt, L. M. (2018). Genetics and Pathogenesis of Diffuse Large B-Cell Lymphoma. New England Journal of Medicine, 378, 1396-1407.
Waanders, E., Scheijen, B., van der Meer, L. T., van Reijmersdal, S. V., van Emst, L., Kroeze, Y., . . . Kuiper, R. P. (2012). The origin and nature of tightly clustered BTG1 deletions in precursor B-cell acute lymphoblastic leukemia support a model of multiclonal evolution. PLoS Genetics, 8.
Morin R D, et al. Frequent mutation of histone-modifying genes in non-Hodgkin lymphoma. Nature. 2011; 476(7360):298-303. Published 2011 Jul. 27. doi:10.1038/nature10351.
Reddy A, et al. Genetic and Functional Drivers of Diffuse Large B Cell Lymphoma. Cell. 2017; 171(2):481-494.e15. doi:10.1016/j.cell.2017.09.027;
Braggio E, et al. Genome-Wide Analysis Uncovers Novel Recurrent Alterations in Primary Central Nervous System Lymphomas. Clin Cancer Res. 2015; 21(17):3986-3994. doi:10.1158/1078-0432.CCR-14-2116.
Pickering C R, et al. Mutational landscape of aggressive cutaneous squamous cell carcinoma. Clin Cancer Res. 2014; 20(24):6582-6592. doi:10.1158/1078-0432.CCR-14-1768.
Witkiewicz A K et al. Whole-exome sequencing of pancreatic cancer defines genetic diversity and therapeutic targets. Nat Commun. 2015; 6:6744. Published 2015 Apr. 9. doi:10.1038/ncomms7744.
Liang W S, et al. Integrated genomic analyses reveal frequent TERT aberrations in acral melanoma. Genome Res. 2017; 27(4):524-532. doi:10.1101/gr.213348.116.
Diamond E L, et al. Efficacy of MEK inhibition in patients with histiocytic neoplasms. Nature. 2019; 567(7749):521-524. doi:10.1038/s41586-019-1012-y.

1. (canceled)

2. A method of treating a patient suffering form a cancer with a BTG1 inactivation comprising the step of:
Determining in a biological sample obtained from said patient the BTG1 mutation status,
Administering a therapeutically effective amount of MCL-1 inhibitor when the patient carries a BTG1 gene inactivation.

3. The method according to claim 1, wherein the cancer is selected in the group consisting B lymphoid hemopathy, cutaneous squamous cell carcinoma, pancreatic cancer, acral melanoma or histiocytosis.

4. The method according to claim 3, wherein the B lymphoid hemopathy is a B lymphoblastic leukemia or a mature B-cell lymphoma.

5. The method according to claim 4, wherein the mature B-cell lymphoma is a Diffuse Large B-Cell Lymphoma (DLBCL), follicular lymphoma, Waldenstrom macroglobulinemia, mantle cell lymphoma, marginal zone B-cell lymphoma, Burkitt lymphoma, hairy cell leukemia or chronic lymphocytic leukemia.

6. The method according to claim 5, wherein the mature B-Cell lymphoma is a Diffuse Large B Cell Lymphoma (DLBCL).

7. The method according to claim 6, wherein the DLBCL is a Germinal Center Derived Lymphoma subtype.

8. The method according to claim 6, wherein the DLBCL is an Activated B-cell subtype.

9. The method according to claim 6, wherein the DLBCL subtype is a MCD/C5 subtype.

10. The method according to claim 1, wherein the BTG1 inactivation results from a mutation selected in Table 2.

11. The method according to claim 1, wherein the BTG1 inactivation results from a missense mutation selected in the group consisting in L26P, G66D and/or I115V.

12. The method according to claim 1, wherein the BTG1 inactivation impairs BTG1 interaction with CAF1 or PRMT1.

13. The method according to claim 1, wherein the MCL-1 inhibitor is S63845, MIK665, ABBV-467, S64315, PRT1419, AMG397, AMG176, and/or AZD5991.

14. The method according to claim 1, wherein the MCL-1 inhibitor is marinopyrrole A, VU661013, A-1210477, BL-08040, omacetaxine mepesuccinate, UMI77 and/or CYC202.
